(19) United States
(12) Patent Application Publication
(10) Pub. No.: US 2015/0336977 A1
(43) Pub. Date: Nov. 26, 2015

(54) Coumestan, Coumestrol, Coumestan Derivatives and Processes of Making the Same and Uses of Same

(71) Applicant: B.G. NEGEV TECHNOLOGIES AND APPLICATIONS LTD., Beer Sheva (IL)

(72) Inventors: Doron Pappo, Beer Sheva (IL); Regev Parnes, Yavne (IL); Umesh Achyutrao Kshirsagar, Beer Sheva (IL); Rivka Ofir, Moshav Hazeva (IL)

(21) Appl. No.: 14/759,641

(22) PCT Filed: Jan. 5, 2014
(86) PCT No.: PCT/IL2014/050009

§ 371 Date: Jul. 7, 2015

Related U.S. Application Data
(60) Provisional application No. 61/749,439, filed on Jan. 7, 2013.
(60) Provisional application No. 61/749,443, filed on Jan. 7, 2013.

(51) Int. Cl. C07D 493/04

(52) U.S. Cl. CPC C07D 493/04

(57) ABSTRACT

The present invention provides new coumestans compounds and processes for the preparation of coumestans, pharmaceutical compositions having a coumestan as an active pharmaceutical ingredient, and methods of utilizing coumestans as selective estrogen receptor modulators (SERMs) for treating estrogen dependent diseases such as breast cancer.

### FIELD OF INVENTION

This invention is directed to, inter alia, coumestans and coumestan derivatives, process of making coumestans and coumestan derivatives and their utilization as selective estrogen receptor modulators (SERMs).

### BACKGROUND OF THE INVENTION

Breast cancer is the most common cancer (excluding non-melanoma skin cancers) among women and the leading cause of cancer deaths in the world. The International Agency for Research on Cancer (IARC) reported that in 2008 around 1.4 million incidence of women diagnosed with breast cancer whereas 39% of these cases resulted in mortality. These facts emphasize the urgent need to develop a strategy not only to treat but also to prevent breast cancer to control the disease and increase survival. One strategy for treating hormone-dependent breast cancer is to inhibit estrogen from binding to its main target the estrogen receptor on tumor cells using selective estrogen receptor modulators (SERMs) such as tamoxifen.

The estrogen receptors (ERα and ERβ) belong to the nuclear hormone family of intracellular receptors, and has essential role in development and maintenance of normal sexual and reproductive function but also in the progression of cancer and other diseases. Tamoxifen, raloxifen have the potential ability to antagonize the detrimental effects of estrogen on breast tissue while producing estrogen-like effects on other systems, however these first generations drugs lack the ability to distinguish between the ER subtypes, a property which could improve their side effect profile. Indeed, much effort is invested to develop such selective ligands (Phillips, C.; Roberts, L. R.; Schade, M.; Bazin, R.; Bent, A.; Davies, N. L.; Moore, R.; Pannifer, A. D.; Pickford, A. R.; Prior, S. H.; Read, C. M.; Scott, A.; Brown, D. G.; Xu, B.; Irving, S. L. Journal of the American Chemical Society 2011, 133, 9696.).

Coumestrol is the most important member of the coumestans family of phytochemicals containing a 6H-benzofuro[3,2-c][1]benzopyran-6-one skeleton. The group comprises hundreds of members that differ in their pattern of oxygenation. The coumestans are found in many plant species and are commonly used in traditional medicine and show a variety of biological activity, including estrogenic, antibacterial, antifungal, snake anti-venom activity and phytoalexine effects (Gaido, K. W.; Leonard, L. S.; Lovell, S.; Gould, J. C.; Babai, D.; Portier, C. J.; McDonnell, D. P. Toxicol. Appl. Pharmacol. 1997, 143, 205; (b) Li, C. C.; Xie, Z. X.; Zhang, Y. D.; Chen, J. H.; Yang, Z. The Journal of Organic Chemistry 2003, 68, 8500.). Coumestrol is an important dietary ingredient present in alfalfa, cabbage and soybeans and its role in human nutrition was studied comprehensively. Due to its potent estrogenic activity coumestrol plays a pivotal role in both the development and progression of breast cancer, (Makela, S.; Davis, V. L.; Tally, W. C.; Korkman, J.; Salo, L.; Vihko, R.; Santti, R.; Korach, K. S. Environ Health Perspect 1994, 102, 572) in the stimulation of bone mineralization (Tsutsumi, N. Biol. Pharm. Bull. 1995, 18, 1012) and in the prevention of bone restoration (Ye, S. F.; Saga, I.; Ichimura, K.; Nagai, T.; Shinoda, M.; Matsuzaki, S. Endocrine Regulations 2003, 37, 145). However, despite coumestrol important medicinal profile the absence of an efficient synthetic strategy that can provide the natural product and its unnatural analogues in a sufficient amount for biology studies frustrated any further developments.

### SUMMARY OF THE INVENTION

In one embodiment, the present invention provides a compound represented by formula XI:

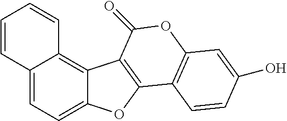 [SMILES: O=c1oc2cc(O)ccc2c2oc3ccc4ccccc4c3c12]

or a pharmaceutically acceptable salt thereof.

In another embodiment, the present invention further provides a compound represented by formula XII:

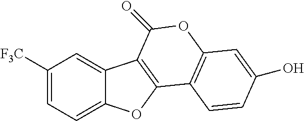 [SMILES: O=c1oc2cc(O)ccc2c2oc3ccc(C(F)(F)F)cc3c12]

or a pharmaceutically acceptable salt thereof.

In another embodiment, the present invention further provides a method for inhibiting mitosis in an estrogen dependent cancer cell, comprising contacting the cell with: a compound of formula XII, a compound of formula XI or their combination.

In another embodiment, the present invention further provides a method for treating a subject afflicted with an estrogen dependent cancer, comprising administering to the subject a pharmaceutical composition comprising a compound of formula XII, a compound of formula XI or their combination.

In another embodiment, the present invention further provides a method for treating a subject afflicted with elevated cholesterol and triglycerides levels, comprising administering to the subject a pharmaceutical composition comprising a compound of formula XII, a compound of formula XI or their combination.

In another embodiment, the present invention further provides a method for selectively modulating an estrogen receptor in a cell, comprising contacting the cell with a compound represented by the following formula:

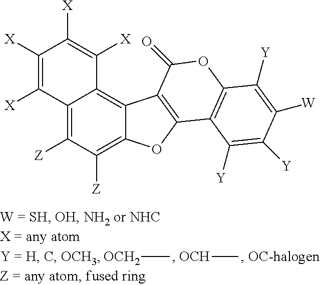

or a pharmaceutically acceptable salt thereof, thereby inhibiting mitosis of an estrogen dependent cancer cell.

In another embodiment, the present invention further provides a method for selectively modulating an estrogen receptor in a cell, comprising contacting the cell with a compound represented by the following formula:

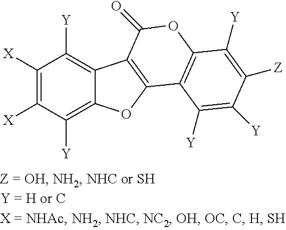

or a pharmaceutically acceptable salt thereof, thereby inhibiting mitosis of an estrogen dependent cancer cell.

In another embodiment, the present invention further provides a method for selectively modulating an estrogen receptor in a cell, comprising contacting the cell with a compound represented by the following formula:

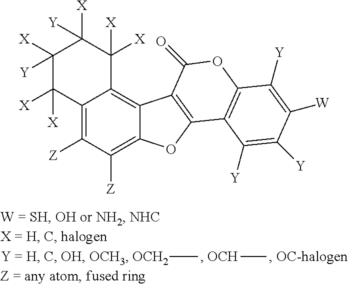

or a pharmaceutically acceptable salt thereof, thereby inhibiting the cell division of an estrogen dependent cancer cell

In another embodiment, the present invention further provides a method for selectively modulating an estrogen receptor in a cell, comprising contacting the cell with a compound represented by the following formula:

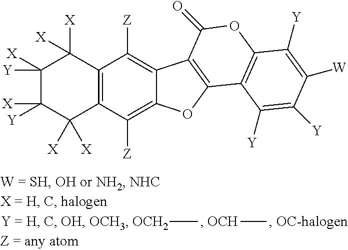

or a pharmaceutically acceptable salt thereof, thereby inhibiting the cell division of an estrogen dependent cancer cell

In another embodiment, the present invention further provides a process for the preparation of a compound of formula I:

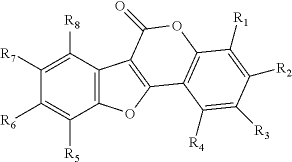

### Wherein:

R₁, R₃, R₄, R₅ and R₈ each independently represent H, C, or a halogen;
R₂ represents Oalkyl, OS(O)₂, OH, H, N or a halogen;
R₆ represents O, H, C, N or C(O)N, alkyl-NH, S(O)NH, AcNH; and
R₇ represents O, H, C, N, C(O)N, alkyl-NH, S(O)NH, AcNH, CO₂Et, CF₃ or a halogen;
said process comprising lactonization of a deprotected benzofuran of formula II:

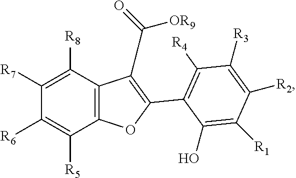

### Wherein:

R₁, R₃, R₄, R₅ and R₈ each independently represent H, C, or a halogen C;
R₂ represents Oalkyl, OS(O)₂, OH, H, N or a halogen;
R₆ represents O, H, C, N or C(O)N, alkyl-NH, S(O)₂NH, S(O)NH, or AcNH;
R₇ represents O, H, C, N, C(O)N, alkyl-NH, S(O)₂NH, S(O)NH, AcNH, CO₂Et, CF₃ or a halogen; and R₉ represents H or C, CH₃, C₂H₅; thereby preparing a compound of formula I.

In another embodiment, the present invention further provides a process for the preparation of a compound of formula III:

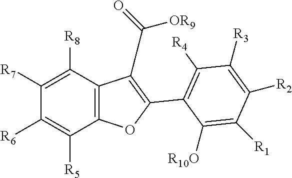

### Wherein:

R₁, R₃, R₄, R₅ and R₈ each independently represent H, C, or a halogen;
R₂ represents Oalkyl, OS(O)₂C, OH, H, N or a halogen;
R₆ represents O, H, C, N or C(O)N, alkylNH, S(O)₂NH, S(O)NH, AcNH; and
R₇ represents O, H, C, N, C(O)N, alkylNH, S(O)₂NH, S(O)NH, AcNH, CO₂Et, CF₃ or a halogen;
R₉ represents H or C, CH₃, C₂H₅; and
R10 represents C, S, Si;
said process comprising mixing ethyl 2-(2,4-dimethoxybenzoyl)acetate and 3-methoxyphenol in 1,2-dichloroethane in the presence of FeCl₃ under air atmosphere or oxygen atmosphere, thereby preparing a compound of formula III. In another embodiment, the present invention further provides that deprotected benzofuran is obtained by contacting a benzofuran of formula III:

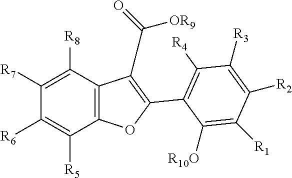

### Wherein:

R₁, R₃, R₄, R₅ and R₈ each independently represent H, C, or a halogen;
R₂ represents Oalkyl, OS(O)₂C, OH, H, N or a halogen;
R₆ represents O, H, C, N or C(O)N, alkylNH, S(O)₂NH, S(O)NH, AcNH; and
R₇ represents O, H, C, N, C(O)N, alkylNH, S(O)₂NH, S(O)NH, AcNH, CO₂Et, CF₃ or a halogen;
R₉ represents H or C, CH₃, C₂H₅;
R10 represents C, S, Si, with a deprotecting solution/agent. In another embodiment, the present invention further provides that the benzofuran of formula III is obtained by iron catalyzed oxidative cross coupling reaction between a compound of formula IV:

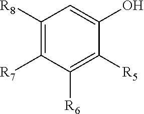

and a compound of formula V:

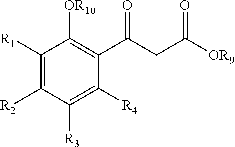

wherein:

R₁, R₃, R₄, R₅ and R₈ each independently represent H, C, or a halogen;

R₂ represents H, OMe, or a halogen;

R₆ represents OMe, H, C, N or AcNH;

R₇ represents OMe, H, C, N, AcNH, CO₂Et, CF₃ or a halogen;

R₉ represents C or H; and

R₁₀ represents H or C.

In another embodiment, the present invention further provides a product comprising formula VI:

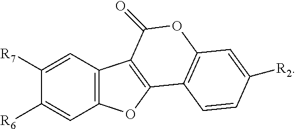

In another embodiment, the present invention further provides a compound of: formula VII:

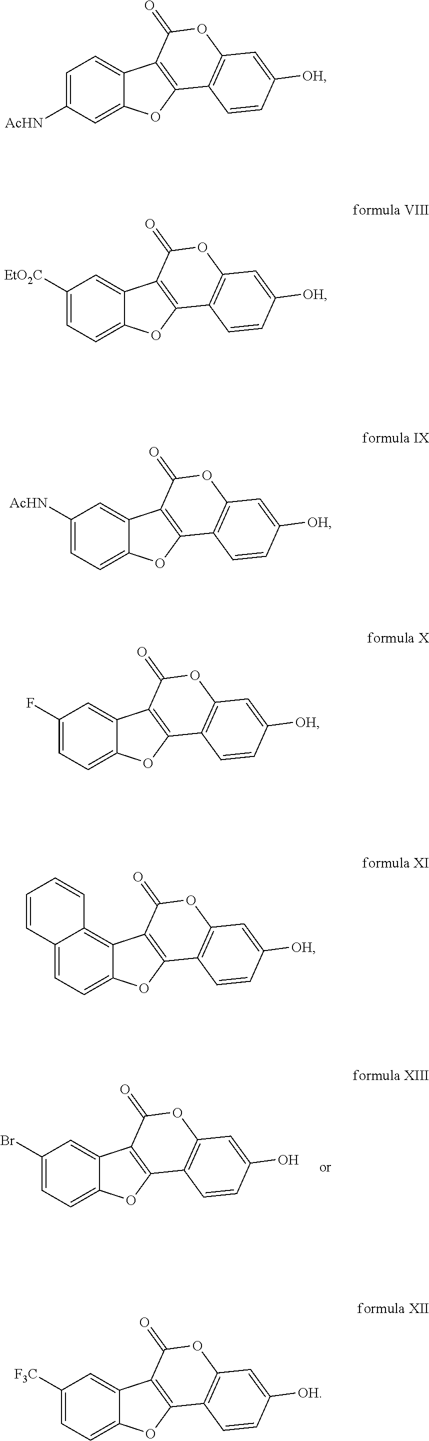

### BRIEF DESCRIPTION OF THE DRAWINGS

FIG. 1. A scheme showing a general retrosynthetic analysis of coumestans.

FIG. 2. A scheme showing the general synthesis of coumestrol.

FIG. 3. A schematic representation showing the interactions between coumestrol and ER-LBD (1U9E). Left, overall structure in ribbon representation of ER-LBD with modeled coumestrol (spheres). Right, zoom in view of two possible coumestrol binding modes that are dependent on the hydroxyl location. (a) Coumestrol (sticks) is directed by the 3-hydroxy group. (b) Coumestrol (pink sticks) is directed by the 9-hydroxy. ER-LBD is represented as green sticks and ribbons.

FIG. 4. A bar graph showing the proliferative effect of selected compounds on estrogen-dependent (MCF-7) and estrogen-independent (MDA-MB-231) cells at 10⁻⁷ M after 6 days in culture.

FIG. 5. A scheme showing the formation of benzofuran 7n (5A), and a practical column-free synthesis of coumestrol under air/oxygen atmospheric conditions (5B).

### DETAILED DESCRIPTION OF THE INVENTION

### Compounds

In one embodiment, the present invention further includes compounds as further described hereinbelow. In another embodiment, the present invention provides that a compound or a product as described herein includes its pharmaceutically acceptable salt. In another embodiment, the present invention further includes any compound obtained by the processes as described hereinabove. In another embodiment, the present invention further includes a compound or a product of formula VI

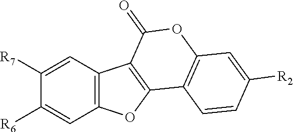

that can be obtained by the processes described hereinabove, wherein: R₂ represents OH; R₆ represents H, or AcNH; and R₇ represents H, AcNH, CO₂Et, F, Br, a halogen, or CF₃. In another embodiment, R₇ represents H, AcNH, CO₂Et, F, or CF₃. In another embodiment, R₇ represents H, AcNH, CO₂Et, F, Br or CF₃. In another embodiment, the present invention further includes a compound or a product of formula VII:

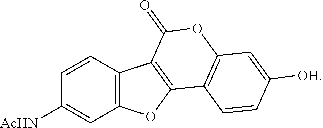 [SMILES: CC(=O)Nc1ccc2c(c1)oc1c3ccc(O)cc3oc(=O)c21]

In another embodiment, the present invention further includes a compound or a product of formula VIII:

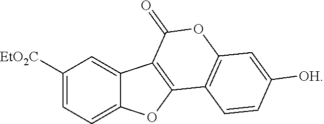 [SMILES: CCOC(=O)c1ccc2oc3c4ccc(O)cc4oc(=O)c3c2c1]

In another embodiment, the present invention further includes a compound or a product of formula IX:

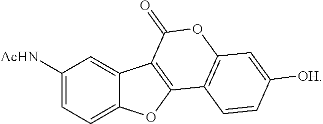 [SMILES: CC(=O)Nc1ccc2oc3c4ccc(O)cc4oc(=O)c3c2c1]

In another embodiment, the present invention further includes a compound or a product of formula X:

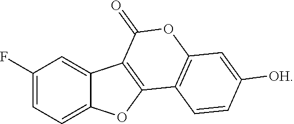 [SMILES: O=c1oc2cc(O)ccc2c2oc3ccc(F)cc3c12]

In another embodiment, the present invention further includes a compound or a product of formula XI:

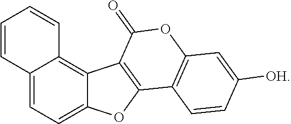 [SMILES: O=c1oc2cc(O)ccc2c2oc3ccc4ccccc4c3c12]

In another embodiment, the present invention further includes a compound or a product of formula XII:

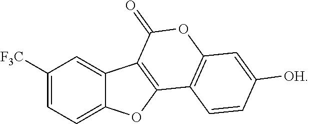 [SMILES: O=c1oc2cc(O)ccc2c2oc3ccc(C(F)(F)F)cc3c12]

In another embodiment, the present invention further includes a compound or a product of formula XIII:

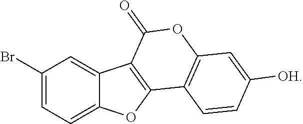 [SMILES: O=c1oc2cc(O)ccc2c2oc3ccc(Br)cc3c12]

In another embodiment, the present invention further includes a pharmaceutical composition comprising a product as described herein and a pharmaceutically acceptable excipient. In another embodiment, the present invention further includes the use of a product of a process of the invention for the preparation of a medicament for selectively modulating an estrogen receptor in a cell. In another embodiment, the present invention further includes the use of a product of any one of formulas as described herein for the preparation of a medicament for selectively modulating an estrogen receptor in a cell.

In one embodiment, the present invention provides a compound or a product represented by formula XIV:

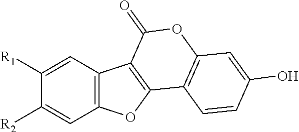

Wherein R₁ is NHAc or H; R₂ is NHAc, H, F, CF₃, or CO₂Et, with the condition that if R₁ is NHAc then R₂ can be only H. In one embodiment, the present invention provides a compound or a product represented by formula XIII:
In another embodiment, the present invention provides a compound or a product represented by formula XV:

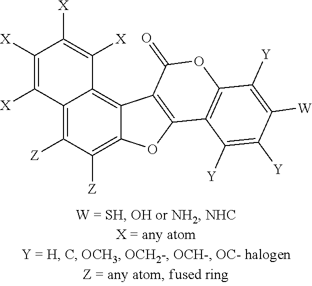

or a pharmaceutically acceptable salt thereof. In another embodiment, the present invention provides a compound or a product represented by formula XVI:

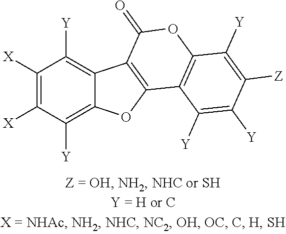

or a pharmaceutically acceptable salt thereof. In another embodiment, the present invention provides a compound or a product represented by formula XVII:

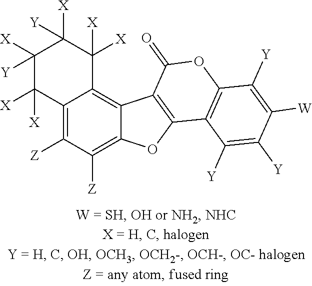

or a pharmaceutically acceptable salt thereof.

In another embodiment, the present invention provides a compound represented by formula XVIII:

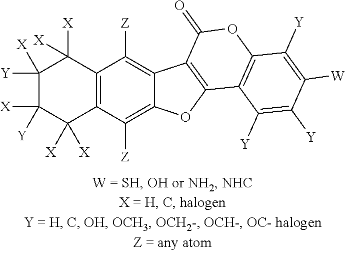

or a pharmaceutically acceptable salt thereof.

In another embodiment, compounds or products of the invention include pharmaceutically acceptable salts, prodrugs, active metabolites and pharmaceutically acceptable solvates thereof of the compounds disclosed herein that are estrogen receptor modulators. In another embodiment, the compounds described herein are estrogen receptor degraders. In another embodiment, the compounds described herein are estrogen receptor antagonists. In another embodiment, the compounds described herein are estrogen receptor agonists. In another embodiment, the compounds described herein are estrogen receptor antagonists in certain tissues and estrogen receptor agonists in other tissues. In another embodiment, the compounds described herein are utilized to treat a patient suffering from estrogen dependent breast cancer. In another embodiment, the compounds described herein are estrogen receptor degraders and estrogen receptor antagonists with minimal or no estrogen receptor agonist activity. In another embodiment, the 3-hydroxy group in coumestrol confers SERM activity. In another embodiment, the 9-hydroxy group can be replaced.

In another embodiment, the compounds described herein are in amorphous forms. In another embodiment, the compounds described herein are in crystalline forms. In another embodiment, compounds described herein are in the form of pharmaceutically acceptable salts. As well, active metabolites of these compounds having the same type of activity are included, in some embodiments, in the scope of the present disclosure. In another embodiment, the compounds described herein exist in an unsolvated form. In another embodiment, the compounds described herein exist in a solvated form. In another embodiment, the compounds described herein are mixed with pharmaceutically acceptable solvents such as water, ethanol, and the like.

In another embodiment, compounds described herein are prepared as prodrugs. A “prodrug” refers to an agent that is converted into the parent drug in vivo. In another embodiment, the design of a prodrug increases the effective water solubility. An example, without limitation, of a prodrug is a compound described herein, which is administered as an ester (the “prodrug”) but then is metabolically hydrolyzed to provide the active entity. In certain embodiments, upon in vivo administration, a prodrug is chemically converted to the biologically, pharmaceutically or therapeutically active form of the compound.

In another embodiment, protected derivatives of the disclosed compound also are contemplated. A variety of suitable for use with the disclosed compounds is disclosed in Greene and Wuts Protective Groups in Organic Synthesis; 3rd Ed.; John Wiley & Sons, New York 1999.

In another embodiment, the compounds described herein are labeled isotopically (e.g. with a radioisotope) or by another other means, including, but not limited to, the use of chromophores or fluorescent moieties, bioluminescent labels, or chemiluminescent labels.

In another embodiment, the compounds are formulated in a pharmaceutically acceptable composition which refers to a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

In another embodiment, “pharmaceutically acceptable salt” refers to a formulation of a compound that does not cause significant irritation to an organism to which it is administered and does not abrogate the biological activity and properties of the compound. In another embodiment, pharmaceutically acceptable salts are obtained by reacting a compound of the invention with an acid. Pharmaceutically acceptable salts are also obtained by reacting a compound of the invention with a base to form a salt.

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. In this application, the use of “or” or “and” means “and/or” unless stated otherwise. Furthermore, use of the term “including” as well as other forms, such as “have”, “having”, “include”, “includes,” and “included,” is not limiting. The section headings used herein are for organizational purposes only and are not to be construed as limiting the subject matter described.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In another embodiment, a modulator is an antagonist. In another embodiment, a modulator is a degrader.

The phrase, “the compounds of the invention” is inclusive, in some embodiments, of the phrase: “pharmaceutical compositions comprising the compounds of the invention”.

The compounds described herein, in some embodiments, are “Selective estrogen receptor modulators” or “SERMs” (as used herein, refers to a molecule that differentially modulates the activity of estrogen receptors in different tissues). In another embodiment, a SERM compound of the invention displays ER antagonist activity in some tissues and ER agonist activity in other tissues. In another embodiment, a SERM compound of the invention displays ER antagonist activity in some tissues and minimal or no ER agonist activity in other tissues. In another embodiment, a SERM compound of the invention displays ER antagonist activity in breast tissues, ovarian tissues, endometrial tissues, and/or cervical tissues but minimal or no ER agonist activity in uterine tissues. In another embodiment, the compounds of the invention selectively modulate an estrogen receptor in a cell. In another embodiment, the compounds of the invention have estrogen agonist activity in a cell of a bone tissue. In another embodiment, the compounds of the invention have estrogen antagonist activity in a cell of a breast tissue.

The term “antagonist” as used herein, refers to a compound of the invention that binds to a nuclear hormone receptor and subsequently decreases the agonist induced transcriptional activity of the nuclear hormone receptor.

The term “agonist” as used herein, refers to a compound of the invention that binds to a nuclear hormone receptor and subsequently increases nuclear hormone receptor transcriptional activity in the absence of a known agonist.

The term “inverse agonist” as used herein, refers to a compound of the invention that binds to a nuclear hormone receptor and subsequently decreases the basal level of nuclear hormone receptor transcriptional activity that is present in the absence of a known agonist.

The term “degrader” as used herein, refers to a compound of the invention that binds to a nuclear hormone receptor and subsequently lowers the steady state protein levels of the receptor. In another embodiment, a degrader as described herein lowers steady state estrogen receptor levels by at least 10%, at least 20%, at least 30%, at least 40%, at least 50%, at least 60%, at least 65%, at least 70%, at least 75%, at least 80%, at least 85%, at least 90% or at least 95%.

The term “selective estrogen receptor degrader” or “SERD” as used herein, refers to a compound of the invention that preferentially binds to estrogen receptors versus other receptors and subsequently lowers the steady state estrogen receptor levels.

“Hormone replacement therapy” refers to treatment given in response to reduced or insufficient estrogen production in a subject, for example as seen in menopause. Hormone replacement therapy often is undertaken in response to aging, ovarectomy or premature ovarian failure. Hormone replacement therapy is often used to help treat one or more of the secondary effects associated with estrogen insufficiency, such as osteoporosis, heart disease, hot flushes and mood disorders.

The term “ER-dependent”, as used herein, refers to diseases or conditions that would not occur, or would not occur to the same extent, in the absence of estrogen receptors.

The term “ER-mediated”, as used herein, refers to diseases or conditions that occur in the absence of estrogen receptors but can occur in the presence of estrogen receptors.

The term “ER-sensitive”, as used herein, refers to diseases or conditions that would not occur, or would not occur to the same extent, in the absence of estrogens.

The term “cancer” as used herein refers to an abnormal growth of cells which tend to proliferate in an uncontrolled way and, in some cases, to metastasize (spread). Examples of cancers include, acute lymphoblastic leukemia, acute myeloid leukemia, adrenocortical carcinoma, anal cancer, appendix cancer, astrocytomas, atypical teratoid/rhabdoid tumor, basal cell carcinoma, bile duct cancer, bladder cancer, bone cancer (osteosarcoma and malignant fibrous histiocytoma), brain stem glioma, brain tumors, brain and spinal cord tumors, breast cancer, bronchial tumors, Burkitt lymphoma, cervical cancer, chronic lymphocytic leukemia, chronic myelogenous leukemia, colon cancer, colorectal cancer, craniopharyngioma, cutaneous T-Cell lymphoma, embryonal tumors, endometrial cancer, ependymoblastoma, ependymoma, esophageal cancer, ewing sarcoma family of tumors, eye cancer, retinoblastoma, gallbladder cancer, gastric (stomach) cancer, gastrointestinal carcinoid tumor, gastrointestinal stromal tumor (GIST), gastrointestinal stromal cell tumor, germ cell tumor, glioma, hairy cell leukemia, head and neck cancer, hepatocellular (liver) cancer, hodgkin lymphoma, hypopharyngeal cancer, intraocular melanoma, islet cell tumors (endocrine pancreas), Kaposi sarcoma, kidney cancer, Langerhans cell histiocytosis, laryngeal cancer, leukemia, Acute lymphoblastic leukemia, acute myeloid leukemia, chronic lymphocytic leukemia, chronic myelogenous leukemia, hairy cell leukemia, liver cancer, non-small cell lung cancer, small cell lung cancer, Burkitt lymphoma, cutaneous T-cell lymphoma, Hodgkin lymphoma, non-Hodgkin lymphoma, lymphoma, Waldenstrom macroglobulinemia, medulloblastoma, medulloepithelioma, melanoma, mesothelioma, mouth cancer, chronic myelogenous leukemia, myeloid leukemia, multiple myeloma, nasopharyngeal cancer, neuroblastoma, non-Hodgkin lymphoma, non-small cell lung cancer, oral cancer, oropharyngeal cancer, osteosarcoma, malignant fibrous histiocytoma of bone, ovarian cancer, ovarian epithelial cancer, ovarian germ cell tumor, ovarian low malignant potential tumor, pancreatic cancer, papillomatosis, parathyroid cancer, penile cancer, pharyngeal cancer, pineal parenchymal tumors of intermediate differentiation, pineoblastoma and supratentorial primitive neuroectodermal tumors, pituitary tumor, plasma cell neoplasm/multiple myeloma, pleuropulmonary blastoma, primary central nervous system lymphoma, prostate cancer, rectal cancer, renal cell (kidney) cancer, retinoblastoma, rhabdomyosarcoma, salivary gland cancer, sarcoma, Ewing sarcoma family of tumors, sarcoma, kaposi, Sezary syndrome, skin cancer, small cell Lung cancer, small intestine cancer, soft tissue sarcoma, squamous cell carcinoma, stomach (gastric) cancer, supratentorial primitive neuroectodermal tumors, T-cell lymphoma, testicular cancer, throat cancer, thymoma and thymic carcinoma, thyroid cancer, urethral cancer, uterine cancer, uterine sarcoma, vaginal cancer, vulvar cancer, Waldenstrom macroglobulinemia, Wilms tumor.

The terms “effective amount” or “therapeutically effective amount,” as used herein, refer to a sufficient amount of a compound of the invention being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The term “subject” or “patient” encompasses mammals. Examples of mammals include, but are not limited to humans, chimpanzees, apes, monkeys, cattle, horses, sheep, goats, swine, rabbits, dogs, cats, rats, mice, guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The terms “compound” or “compounds” as used herein, include “product” or “products”, accordingly.

### Cancer

In another embodiment, compounds disclosed herein are estrogen receptor degraders and estrogen receptor antagonists that exhibit: minimal or no estrogen receptor agonism; and/or anti-proliferative activity against breast cancer, ovarian cancer, endometrial cancer, cervical cancer cell lines; and/or maximal anti-proliferative efficacy against breast cancer, ovarian cancer, endometrial cancer, cervical cell lines in-vitro; and/or minimal agonism in the human endometrial (Ishikawa) cell line; and/or no agonism in the human endometrial (Ishikawa) cell line; and/or minimal or no agonism in the immature rat uterine assay in-vivo; and/or inverse agonism in the immature rat uterine assay in-vivo; and/or anti-tumor activity in breast cancer, ovarian cancer, endometrial cancer, cervical cancer cell lines in xenograft assays in-vivo or other rodent models of these cancers.

In another embodiment, compounds disclosed herein are used to inhibit mitosis of an estrogen dependent cancer cell. In another embodiment, the invention provides a method for inhibiting mitosis in an estrogen dependent cancer cell comprising contacting the cell with a compound as disclosed herein. In another embodiment, mitosis according to the invention is aberrant mitosis of a cancerous cell. In another embodiment, mitosis according to the invention is repeated and uncontrolled mitosis. In another embodiment, mitosis according to the invention is repeated and uncontrolled mitosis of an estrogen dependent, transformed, cancer cell. In another embodiment, mitosis is estrogen dependent mitosis.

In another embodiment, compounds or products disclosed herein are used to treat cancer in a mammal. In another embodiment, a method for treating a subject afflicted with an estrogen dependent cancer, comprising administering to the subject a pharmaceutical composition comprising a compound as disclosed herein. In another embodiment, the proliferation or mitosis of a tumor comprising estrogen dependent cancer cells and/or estrogen dependent metastatic cells is inhibited according to the methods of the invention.

In another embodiment, the cancer is breast cancer, ovarian cancer, endometrial cancer, prostate cancer, uterine cancer, cervical cancer or lung cancer. In another embodiment, the cancer is breast cancer. In another embodiment, the cancer is a hormone dependent cancer. In another embodiment, the cancer is an estrogen receptor dependent cancer. In another embodiment, the cancer is an estrogen-sensitive cancer. In another embodiment, the cancer is resistant to anti-hormonal treatment. In another embodiment, the cancer is an estrogen-sensitive cancer or an estrogen receptor dependent cancer that is resistant to anti-hormonal treatment. In another embodiment, anti-hormonal treatment includes treatment with at least one compound of the invention.

In another embodiment, compounds disclosed herein are used to treat hormone receptor positive metastatic breast cancer. In another embodiment, the mammal is a postmenopausal woman. In another embodiment, the mammal is a postmenopausal woman with disease progression following anti-estrogen therapy. In another embodiment, compounds disclosed herein are used to treat cancer in a mammal, wherein the mammal is chemotherapy-naive. In another embodiment, compounds disclosed herein are used to treat cancer in a mammal, wherein the mammal is being treated for cancer with at least one anti-cancer agent. In one embodiment, the cancer is a hormone refractory cancer.

In another embodiment, compounds disclosed herein are used to treat a hormonal dependent benign or malignant disease of the breast or reproductive tract in a mammal. In another embodiment, the benign or malignant disease is breast cancer.

In another embodiment, compounds disclosed herein are used in the treatment of leiomyoma in a mammal. In another embodiment, the leiomyoma is an uterine leiomyoma, esophageal leiomyoma, cutaneous leiomyoma or small bowel leiomyoma. In another embodiment, compounds disclosed herein are used in the treatment of fibroids in a mammal.

### Other Conditions

In another embodiment, compounds of the invention are used to selectively modulate an estrogen receptor in a subject and thus are useful for treating a variety of disorders, including those characterized by an estrogen deficiency. Moreover, because certain disclosed compounds exhibit selectivity for one or more estrogen receptors, the compounds are used to treat conditions including but not limited to those described as autonomic dysfunctions, cognitive decline, motor dysfunctions, mood disorders, eating disorders and cardiovascular disorders, as well as different types of disorders. Generally, the compounds are useful for hormone replacement therapy without inducing the same incidence of serious side effects associated with the steroidal hormones (such as estrogen or synthetic estrogens) used in current hormone replacement therapies. The disclosed compounds also avoid side effects such as hot flushes encountered in treatment with currently known SERMs, such as tamoxifen or raloxifene. In another embodiment, compounds of the invention are used to treat disorders including, without limitation, ischemia-induced neuronal death, head trauma, Alzheimer's disease, disorders of temperature regulation, such as hot flushes, sleep cycle disruptions, Parkinson's disease, tardive diskinesia, depression, schizophrenia, anorexia nervosa, bulimia nervosa, cardiovascular disease, atherosclerosis, long QTL syndromes, such as Romano-Ward or Torsades de Pointes syndromes, osteoporosis, rheumatoid arthritis, osteoarthritis, bone fractures and multiple sclerosis.

In another embodiment, compounds of the invention and pharmaceutical compositions comprising the compounds of the invention are used for treating a subject afflicted with enhanced bone turnover. In another embodiment, compounds of the invention and pharmaceutical compositions comprising the compounds of the invention are used for increasing bone density. In another embodiment, compounds of the invention and pharmaceutical compositions comprising the compounds of the invention are used for reducing the risk of fractures in women with a history of osteoporosis. In another embodiment, bone turnover is postmenopausal osteoporosis.

In another embodiment, compounds of the invention and pharmaceutical compositions comprising the compounds of the invention are used for treating a subject afflicted with elevated cholesterol and triglycerides levels. In another embodiment, compounds of the invention and pharmaceutical compositions comprising the compounds of the invention are used for decreasing low-density cholesterol.

In another embodiment, compounds of the invention are provided for treating or protecting against various conditions and disorders, including conditions that are associated with menopause or other conditions characterized by estrogen insufficiency, such as those associated with ovarectomy, ovarian failure or menopause. Examples of such conditions include, without limitation, hot flushes, cognitive decline, osteoporosis, depression, ischemic brain damage and atherosclerosis. In another embodiment, compounds disclosed herein are used in the treatment of endometriosis in a mammal.

The ability of the disclosed compounds to inhibit or ameliorate hot flushes can be determined, for example, in a standard assay that measures the ability of an agent to blunt the increase in tail skin temperature that occurs when morphine-addicted rats undergo acute withdrawal from morphine using naloxone. See, Merchenthaler, et al. The effect of estrogens and antiestrogens in a rat model for hot flush. Maturitas 1998, 30, 307-316, which is hereby incorporated by reference in its entirety. See also, Berendsen et al. Effect of tibolone and raloxifene on the tail temperature of oestrogen-deficient rats. Eur. J. Pharmacol. 2001, 419, 47-54; and Pan et al.

In another embodiment, compounds of the invention are useful for the treatment of multiple sclerosis. In another embodiment, compounds of the invention are useful for treating eating disorders, such as anorexia nervosa and/or bulimia nervosa can be identified using a simple feeding assay as is known to those of ordinary skill in the art.

In another embodiment, the compounds of the invention are used to treat autoimmune diseases, particularly autoimmune diseases that occur more frequently in women than in men. Examples of such diseases include, without limitation, multiple sclerosis, rheumatoid arthritis, Grave's disease, systemic lupus erythematosus and myasthenia gravis. In another embodiment the disclosed compounds function to maintain or enhance immune competency in a subject. Moreover, the disclosed compounds exert prophylactic effects against certain types of injuries. For example, the compounds can be used as neuroprotectants. Indeed, compounds that agonize the membrane-associated estrogen receptor identified herein act as neuroprotectants in response to ischemic stroke and inhibit reperfusion injury.

Moreover, because of the ability of the disclosed compounds to selectively modulate one or more specific types of estrogen receptor, they can be used to identify the contribution of different estrogen receptors that mediate different physiological effects. The disclosed compounds also can be used to bind to and identify the particular class of membrane bound receptors at which these agents act.

In another embodiment, compounds of the invention are used in affinity chromatography. Because examples of the presently disclosed compounds bind to a novel, membrane-associated estrogen receptor, the compounds can be used to purify the receptor, or remove the receptor from a sample. To use the compounds, they typically are attached to a solid support as is known to those of ordinary skill in the art. The compounds can be attached directly or via a linker molecule.

In another embodiment, use of the compounds of the invention is not limited to conditions involving estrogen insufficiency. Techniques and assays for characterizing the efficacy of therapeutics for treating or preventing such conditions and disorders are well known and are described, for example, by Malamas et al. and Mewshaw et al. in U.S. patent publication numbers 2003/0171412 A1 and 2003/0181519 A1, respectively. Both the Malamas et al. and Mewshaw et al. publications are incorporated by reference in their entireties.

### Routes of Administration

Suitable routes of administration include, but are not limited to, oral, parenteral (e.g., intravenous, subcutaneous, intramuscular), intranasal, buccal, topical, rectal, aerosol, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections. In certain embodiments, a compound as described herein is administered in a systemic manner. In certain other embodiments, a compound as described herein is administered in a local rather than systemic manner.

### Compositions/Formulations

In another embodiment, the compounds described herein are formulated into pharmaceutical compositions. Pharmaceutical compositions of the invention are formulated in a conventional manner using one or more pharmaceutically acceptable inactive ingredients that facilitate processing of the active compounds into preparations that are used pharmaceutically. A formulation depends upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In another embodiment, a pharmaceutical composition comprises a mixture of a compound of the invention and at least one additional active ingredient. In another embodiment, a pharmaceutical composition comprises inactive ingredients, such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition, in some embodiments, facilitates administration of the compound to a mammal.

In another embodiment, a pharmaceutical composition comprises a compound of the invention, and/or a pharmaceutically acceptable salt thereof, as an active ingredient in free-acid or free-base form, or in a pharmaceutically acceptable salt form. In another embodiment, the pharmaceutical compositions described herein include the use of N-oxides (if appropriate), crystalline forms, amorphous phases, as well as active metabolites of these compounds having the same type of activity.

In another embodiment, pharmaceutical compositions described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, enteric coated formulations, pulsatile release formulations, multiparticulate formulations, and mixed immediate and controlled release formulations.

In another embodiment, the compound of the invention, or a pharmaceutically acceptable salt thereof, is administered systemically. In another embodiment, the compound of the invention, or a pharmaceutically acceptable salt thereof, is administered orally. All formulations for oral administration are in dosages suitable for such administration. In another embodiment, the solid dosage forms disclosed herein are in the form of a tablet, a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. In another embodiment, the pharmaceutical formulation is in the form of a tablet. In another embodiment, the pharmaceutical formulation is in the form of a suspension tablet, a fast-melt tablet, a bite-disintegration tablet, a rapid-disintegration tablet, an effervescent tablet, or a caplet. In another embodiment, pharmaceutical formulation is in the form of a capsule.

In another embodiment, the pharmaceutical solid oral dosage forms are formulated to provide a controlled release of the active compound. Controlled release profiles include, for example, sustained release, prolonged release, pulsatile release, and delayed release profiles.

In another embodiment, liquid formulation dosage forms for oral administration are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002).

In another embodiment, for buccal or sublingual administration, the compositions optionally take the form of tablets, lozenges, or gels formulated in a conventional manner.

In another embodiment, a compound of the invention, or a pharmaceutically acceptable salt thereof, is formulated into a pharmaceutical composition suitable for intramuscular, subcutaneous, or intravenous injection. Parenteral injections involve either bolus injection and/or continuous infusion.

In another embodiment, a compound of the invention, or a pharmaceutically acceptable salt thereof, is administered intravenously. In another embodiment, a compound of the invention, or a pharmaceutically acceptable salt thereof, is administered subcutaneously.

In another embodiment, a compound of the invention, or a pharmaceutically acceptable salt thereof, is administered topically. In such embodiments, a compound of the invention, or a pharmaceutically acceptable salt thereof, is formulated into a variety of topically administrable compositions, such as solutions, suspensions, lotions, gels, pastes, shampoos, scrubs, rubs, smears, medicated sticks, medicated bandages, balms, creams or ointments. In another embodiment, a compound of the invention, or a pharmaceutically acceptable salt thereof, is administered topically to the skin of mammal. In another embodiment, a compound of the invention is prepared as a transdermal dosage form.

In another embodiment, the use of a compound of the invention, or a pharmaceutically acceptable salt thereof, in the manufacture of a medicament for treating a disease, disorder or conditions in which the activity of estrogen receptors contributes to the pathology and/or symptoms of the disease or condition. In another embodiment, the disease or condition is any of the diseases or conditions specified herein.

### Dosing

In one embodiment, the compound of the invention, or a pharmaceutically acceptable salt thereof, is used in the preparation of medicaments for the treatment of diseases or conditions in a mammal that would benefit from a reduction of estrogen receptor activity. Methods for treating any of the diseases or conditions described herein in a mammal in need of such treatment, involves administration of pharmaceutical compositions that include the compound of the invention, or a pharmaceutically acceptable salt, N-oxide, active metabolite, prodrug, or pharmaceutically acceptable solvate thereof, in therapeutically effective amounts to the mammal.

Therapeutically effective amounts depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In any of the method of treatments described herein, the effective amount of the compound of the invention is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In one embodiment, the methods of treatment comprise single administration of the effective amount of the compound, including further embodiments in which (i) the compound is administered once; (ii) the compound is administered to the mammal multiple times over the span of one day; (iii) continually; or (iv) continuously.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain embodiments wherein the patient's condition does not improve, upon the doctor's discretion the compound is administered chronically, that is, for an extended period of time.

In certain embodiments wherein a patient's status does improve, the dose of drug being administered is temporarily reduced or temporarily suspended for a certain length of time (i.e., a “drug holiday”).

In another embodiment, doses employed for adult human treatment are typically in the range of 0.01 mg-5000 mg per day. In another embodiment, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day. In one embodiment, the daily dosages appropriate for the compound of the invention, or a pharmaceutically acceptable salt thereof, described herein are from about 0.01 to about 50 mg/kg per body weight.

### Combinations

In another embodiment, it is appropriate to administer a compound of the invention, or a pharmaceutically acceptable salt thereof, in combination with one or more other therapeutic agents. In certain embodiments, the pharmaceutical composition further comprises one or more anti-cancer agents. In certain embodiments, the pharmaceutical composition further comprises an additional SERM.

In another embodiment, a compound of the invention, or a pharmaceutically acceptable salt thereof, is co-administered with a second therapeutic agent, wherein the compound of the invention, or a pharmaceutically acceptable salt thereof, and the second therapeutic agent modulate different aspects of the disease, disorder or condition being treated, thereby providing a greater overall benefit than administration of either therapeutic agent alone.

In another embodiment, methods for treatment of estrogen receptor-dependent or estrogen receptor-mediated conditions or diseases, such as proliferative disorders, including cancer, comprises administration to a mammal a compound of the invention, or a pharmaceutically acceptable salt thereof, in combination with at least one additional therapeutic agent.

In another embodiment, a compound of the invention, or a pharmaceutically acceptable salt thereof, in combination with hormone blocking therapy, chemotherapy, radiation therapy, monoclonal antibodies, or combinations thereof.

In another embodiment, a compound of the invention, or a pharmaceutically acceptable salt thereof, is used in combination with anti-emetic agents to treat nausea or emesis, which result from the use of a compound of the invention, anti-cancer agent(s) and/or radiation therapy.

In another embodiment, a compound of the invention, or a pharmaceutically acceptable salt thereof, is used in combination with an agent useful in the treatment of anemia or neutropenia.

In another embodiment, a compound of the invention, or a pharmaceutically acceptable salt thereof, is administered with corticosteroids. In another embodiment, a compound of the invention, or a pharmaceutically acceptable salt thereof, is co-administered with an analgesic.

In another embodiment, a compound of the invention, or a pharmaceutically acceptable salt thereof, is used in combination with radiation therapy. In one embodiment, a disclosed SERM is used in combination with additional compounds disclosed herein and/or other therapeutic agents, such as other SERMs, anti-cancer agents or anti-proliferative agents. For example the disclosed compounds may be used with chemotherapeutic agents, such as tamoxifen, taxol, epothilones, methotrexate, and the like. In one aspect, a disclosed SERM is used in combination with a steroid hormone, such as an estrogen, including 17-beta-estradiol, a progesterone or the like. The estrogen or progesterone can be a naturally occurring or synthetic estrogen or progesterone. When different therapeutic agents are used in combination, the therapeutic agents can be administered together or separately. The therapeutic agents can be administered alone, but more typically are administered with a pharmaceutical carrier selected on the basis of the chosen route of administration and standard pharmaceutical practice.

### Synthesis

In another embodiment, compounds of the invention are synthesized according to the methods of (Emerson, O. H.; Bickoff, E. M. Journal of the American Chemical Society 1958, 80, 4381; Al-Maharik, N.; Botting, N. P. Tetrahedron 2004, 60, 1637; Yao, T.; Yue, D.; Larock, R. C. J. Org. Chem. 2005, 70, 9985; Kraus, G. A.; Zhang, N. J. Org. Chem. 2000, 65, 5644; Hiroya, K.; Suzuki, N.; Yasuhara, A.; Egawa, Y.; Kasano, A.; Sakamoto, T. J. Chem. Soc., Perkin Trans. 1 2000, 4339; Pandit, S. B.; Gadre, S. Y. Synth. Commun. 1988, 18, 157; Kappe, T.; Laschober, R. Synthesis 1990, 387; Tang, L.; Pang, Y.; Yan, Q.; Shi, L.; Huang, J.; Du, Y.; Zhao, K. J. Org. Chem. 2011, 76, 2744; Chang, C.-F.; Yang, L.-Y.; Chang, S.-W.; Fang, Y.-T.; Lee, Y.-J. Tetrahedron 2008, 64, 3661; Gong, D.-H.; Li, C.-Z.; Yuan, C.-Y. Chin. J. Chem. 2001, 19, 522; da, S. A. J. M.; Melo, P. A.; Silva, N. M. V.; Brito, F. V.; Buarque, C. D.; de, S. D. V.; Rodrigues, V. P.; Pocas, E. S. C.; Noel, F.; Albuquerque, E. X.; Costa, P. R. R. Bioorg. Med. Chem. Lett. 2001, 11, 283; Rani, B. S. U.; Darbarwar, M. J. Indian Chem. Soc. 1986, 63, 1060; Darbarwar, M.; Sundaramurthy, V.; Rao, N. V. S. Proc. Indian Acad. Sci., Sect. A 1974, 80, 93; Deschamps-Vallet, C.; Mentzer, C. Compt. rend. 1960, 251, 736; Wanzlick, H. W.; Gritzky, R.; Heidepriem, H. Chem. Ber. 1963, 96, 305; Tang, L.; Pang, Y.; Yan, Q.; Shi, L.; Huang, J.; Du, Y.; Zhao, K. The Journal of Organic Chemistry 2011, 76, 2744. All of which are incorporated by reference in their entirety.).

In one embodiment, the present invention provides a process for the preparation of a compound of formula I:

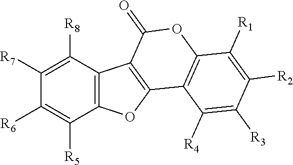

### Wherein:

R₁, R₂, R₃, R₄, R₅ R₆, R₇, and R₈ each independently represent: H, C, halogen, alkyl, cycloalkyl, O, N, Oalkyl, OS(O)₂, C(O)N, alkyl-NH, S(O)NH, AcNH, CO₂Et, or CF₃, wherein the process comprises lactonization of a deprotected benzofuran. In one embodiment, the present invention provides a compound or a product of formula I. In one embodiment, the present invention provides the use of a compound or a product of formula I according to the methods as described herein.

In one embodiment, the present invention provides a process for the preparation of a compound of formula I:

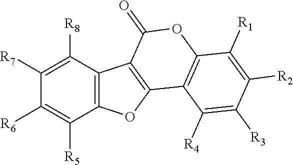

### Wherein:

R₁, R₃, R₄, R₅ and R₈ each independently represent: H, C, halogen, alkyl, cycloalkyl, O, N, Oalkyl, OS(O)₂, C(O)N, alkyl-NH, S(O)NH, AcNH, CO₂Et, or CF₃,
R₂ represents Oalkyl, OS(O)₂, OH, H, N or a halogen;
R₆ represents O, H, C, N or C(O)N, alkyl-NH, S(O)NH, AcNH; and
R₇ represents O, H, C, N, C(O)N, alkyl-NH, S(O)NH, AcNH, CO₂Et, CF₃ or a halogen, wherein the process comprises lactonization of a deprotected benzofuran.

In another embodiment, the present invention provides a process for the preparation of a compound of formula I, wherein R₁, R₃, R₄, R₅ and R₈ each independently represent any atom and R₂ represents Oalkyl, OS(O)₂, OH, H, N or a halogen; R₆ represents O, H, C, N or C(O)N, alkyl-NH, S(O)NH, AcNH, or a halogen; and R₇ represents O, H, C, N, C(O)N, alkyl-NH, S(O)NH, AcNH, CO₂Et, CF₃ or a halogen, wherein the process comprises lactonization of a deprotected benzofuran.

In another embodiment, the present invention provides a process for the preparation of a compound of formula I,

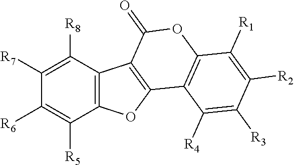

### Wherein:

R₁, R₃, R₄, R₅ and R₈ each independently represent H or C;
R₂ represents Oalkyl, OS(O)₂, OH, H, N or a halogen;
R₆ represents O, H, C, N or C(O)N, alkyl-NH, S(O)NH, AcNH; and
R₇ represents O, H, C, N, C(O)N, alkyl-NH, S(O)NH, AcNH, CO₂Et, CF₃ or a halogen; wherein the process comprises lactonization of a deprotected benzofuran.

In another embodiment, a deprotected benzofuran comprises formula II:

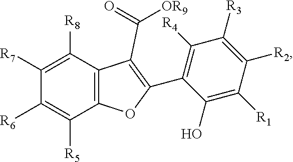

### Wherein:

R₁ to R₉ each independently represent: H, C, halogen, alkyl, cycloalkyl, O, N, Oalkyl, OS(O)₂, C(O)N, alkyl-NH, S(O)NH, AcNH, CO₂Et, or CF₃. In another embodiment, a deprotected benzofuran comprises formula II, wherein R₁, R₃, R₄, R₅ and R₈ independently any atom; R₂ represents Oalkyl, OS(O)₂, OH, H, N or a halogen; R₆ represents O, H, C, N or C(O)N, alkyl-NH, S(O)₂NH, S(O)NH, AcNH or a halogen; R₇ represents O, H, C, N, C(O)N, alkyl-NH, S(O)₂NH, S(O)NH, AcNH, CO₂Et, CF₃ or a halogen; and R₉ represents H or C, CH₃, C₂H₅. In another embodiment, a deprotected benzofuran comprises formula II, wherein R₁, R₃, R₄, R₅ and R₈ each independently represent H or C; R₂ represents Oalkyl, OS(O)₂, OH, H, N or a halogen; R₆ represents O, H, C, N or C(O)N, alkyl-NH, S(O)₂NH, S(O)NH, AcNH or a halogen; R₇ represents O, H, C, N, C(O)N, alkyl-NH, S(O)₂NH, S(O)NH, AcNH, CO₂Et, CF₃ or a halogen; and R₉ represents H C, CH₃, or C₂H₅.
In one embodiment, the present invention provides a compound of formula I or a salt thereof.
In one embodiment, the present invention provides a compound of formula II or a salt thereof.
In one embodiment, the present invention provides the use of a compound or a product of formula I or formula II according to the methods as described herein.

In another embodiment, the present invention provides that lactonization of a deprotected benzofuran is under condition comprising a temperature above 50° C. In another embodiment, the present invention provides that lactonization of a deprotected benzofuran is under condition comprising a temperature above 60° C. In another embodiment, the present invention provides that lactonization of a deprotected benzofuran is under condition comprising a temperature above 70° C. In another embodiment, the present invention provides that lactonization of a deprotected benzofuran is under condition comprising a temperature above 80° C. In another embodiment, the present invention provides that lactonization of a deprotected benzofuran is under condition comprising a temperature above 90° C. In another embodiment, the present invention provides that lactonization of a deprotected benzofuran is under condition comprising a temperature between 60 to 100° C.

In another embodiment, the present invention provides that lactonization of a deprotected benzofuran is preformed in a solvent. In another embodiment, the present invention provides that lactonization of a deprotected benzofuran is preformed in a polar solvent. In another embodiment, the present invention provides that lactonization of a deprotected benzofuran is preformed in a non-polar solvent. In another embodiment, the present invention provides that lactonization of a deprotected benzofuran is preformed in: ethanol, methanol, acetonitrile, H₂O, or any mixture thereof. In another embodiment, the present invention provides that lactonization of a deprotected benzofuran is preformed in: toluene, any chlorinated solvent, tetrahydrofuran, dioxane, or any mixture thereof. In another embodiment, the present invention provides that lactonization of a deprotected benzofuran is preformed in: toluene, any chlorinated solvent, tetrahydrofuran, dioxane, ethanol, methanol, acetonitrile, H₂O, or any mixture thereof.

In another embodiment, the present invention provides that a deprotected benzofuran is obtained by contacting a benzofuran of formula III:

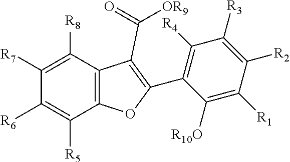

### Wherein:

R₁ to R₁₀ each independently represent: H, C, halogen, alkyl, cycloalkyl, O, N, Oalkyl, OS(O)₂, C(O)N, alkyl-NH, S(O)NH, AcNH, CO₂Et, or CF₃. In another embodiment, the benzofuran comprises formula III, wherein R₁ to R₁₀ are independently any atom. In another embodiment, the benzofuran comprises formula III, wherein R₁ to R₈ are independently any atom; R₉ and R₁₀ are independently alkyl, S(O)₂, or H, CN, alkyl-NH, SONH, SNH, or COEt. In another embodiment, the benzofuran comprises formula III, wherein R₁, R₃, R₄, R₅ and R₈ each independently represent H or C; R₂ represents Oalkyl, OS(O)₂C, OH, H, N or a halogen; R₆ represents O, H, C, N or C(O)N, alkylNH, S(O)₂NH, S(O)NH, AcNH; R₇ represents O, H, C, N, C(O)N, alkylNH, S(O)₂NH, S(O)NH, AcNH, CO₂Et, CF₃ or a halogen; R₉ represents H or C, CH₃, C₂H₅; and R₁₀ represents C, S, Si. In another embodiment, the benzofuran comprises formula III, wherein R₁, R₃, R₄, R₅ and R₈ each independently represent H or C; R₂ represents Oalkyl, OS(O)₂C, OH, H, N or a halogen;
R₆ represents O, H, C, N or C(O)N, alkylNH, S(O)₂NH, S(O)NH, AcNH; R₇ represents O, H, C, N, C(O)N, alkylNH, S(O)₂NH, S(O)NH, AcNH, CO₂Et, CF₃ or a halogen; R₉ represents H or C, CH₃, C₂H₅; and R10 represents C, S, Si. In one embodiment, the present invention provides a compound or a product of formula III. In one embodiment, the present invention provides the use of a compound or a product of formula III according to the methods as described herein.

In another embodiment, the present invention provides that deprotecting benzofuran is contacting a benzofuran having protecting group or groups with a deprotecting solution/agent. Protecting groups and deprotecting solutions/agents are described in Greene and Wuts Protective Groups in Organic Synthesis; 3rd Ed.; John Wiley & Sons, New York (1999) which is hereby incorporated by reference in its entirety.

In one embodiment, the present invention provides a compound of formula III or a salt thereof. In one embodiment, the present invention provides a combination of any two or more compounds as described herein.

In another embodiment, the present invention provides that a benzofuran of formula III is obtained by iron catalyzed oxidative cross coupling reaction between a compound of formula IV:

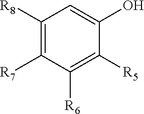

and a compound of formula V:

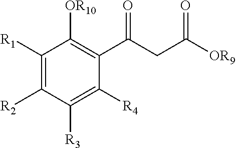

R₁ to R₈ each independently represent H or C; R₂ represents H, OMe, or a halogen; R₆ represents OMe, H, C, N or AcNH; R₇ represents OMe, H, C, N, AcNH, CO₂Et, CF₃ or a halogen; R₉ represents C or H; and R₁₀ represents C or H. In another embodiment, compounds IV and V have R₁, R₃, R₄, R₅ and R₈ each independently represent any atom; R₂ represents C, H, OMe, or a halogen; R₆ represents OMe, H, C, N, a halogen, or AcNH; R₇ represents OMe, H, C, N, AcNH, CO₂Et, CF₃ or a halogen; R₉ represents C or H; and R₁₀ represents C or COH. In one embodiment, the present invention provides a compound of formula IV and/or a compound of formula IV or any salt thereof.

In another embodiment, iron catalyzed oxidative cross coupling reaction is a reaction comprising the presence of iron (II). In another embodiment, iron catalyzed oxidative cross coupling reaction is a reaction comprising the presence of iron (III). In another embodiment, iron catalyzed oxidative cross coupling reaction is a reaction comprising any organic peroxide or oxygen molecule in a chlorinated or hydrocarbon solvent. In another embodiment, iron catalyzed oxidative cross coupling reaction is a reaction comprising FeCl₃, FeCl₃(H₂O)₆, FeCl₂, FeCl₂(H₂O)₄, Fe(ClO₄)₃(H₂O)ₓ, Fe(ClO₄)₂(H₂O)ₓ ditertbutylperoxide, oxygen molecule or any combination thereof. In another embodiment, “x” equals to any number from 1 to 50. In another embodiment, “x” equals to any number from 1 to 5. In another embodiment, “x” equals to any number from 1 to 4. In another embodiment, iron catalyzed oxidative cross coupling reaction is a reaction comprising the presence of iron (II) or iron (III) such as FeCl₃, FeCl₃(H₂O)₆, FeCl₂, FeCl₂(H₂O)₄ or any combination thereof in the presence of N-hydroxyphthalimide.

In another embodiment, iron catalyzed oxidative cross coupling reaction is performed at a temperature of above 35° C. In another embodiment, iron catalyzed oxidative cross coupling reaction is performed at a temperature of above 40° C. In another embodiment, iron catalyzed oxidative cross coupling reaction is performed at a temperature of above 50° C. In another embodiment, iron catalyzed oxidative cross coupling reaction is performed at a temperature of above 60° C. In another embodiment, iron catalyzed oxidative cross coupling reaction is performed at a temperature of above 70° C. In another embodiment, iron catalyzed oxidative cross coupling reaction is performed at a temperature of above 80° C. In another embodiment, iron catalyzed oxidative cross coupling reaction is performed at a temperature of above 90° C. In another embodiment, iron catalyzed oxidative cross coupling reaction is performed at a temperature between 35 to 100° C. In another embodiment, iron catalyzed oxidative cross coupling reaction is performed at a temperature between 40 to 100° C. In another embodiment, iron catalyzed oxidative cross coupling reaction is performed at a temperature between 50 to 100° C. In another embodiment, iron catalyzed oxidative cross coupling reaction is performed at a temperature between 60 to 90° C.

In another embodiment, the present invention provides that a benzofuran of formula III is obtained by mixing ethyl 2-(2,4-dimethoxybenzoyl)acetate (compound 2b in FIGS. 1, 2 and 5) and 3-methoxyphenol (compound 3a in FIGS. 1, 2 and 5) in 1,2-dichloroethane in the presence of FeCl₃ (1-20 mol %) or FeCl₃(H₂O)₆ (1-20 mol %) under air atmosphere or oxygen atmosphere (see FIG. 5). In another embodiment, ethyl 2-(2,4-dimethoxybenzoyl)acetate is in 0.6-1.5 equivalents and 3-methoxyphenol is in 0.8-1.6 equivalents. In another embodiment, ethyl 2-(2,4-dimethoxybenzoyl)acetate is in 0.9-1.1 equivalents and 3-methoxyphenol is in 1-1.2 equivalents. In another embodiment, ethyl 2-(2,4-dimethoxybenzoyl)acetate is in 1 equivalent and 3-methoxyphenol is in 1.1 equivalents. In another embodiment, this reaction carried under air atmosphere or oxygen atmosphere is performed in a temperature of above 50° C. In another embodiment, this reaction carried under air atmosphere or oxygen atmosphere is performed in a temperature of above 60° C. In another embodiment, this reaction carried under air atmosphere or oxygen atmosphere is performed in a temperature of above 70° C. In another embodiment, this reaction carried under air atmosphere or oxygen atmosphere is performed in a temperature of above 80° C. In another embodiment, this reaction carried under air atmosphere or oxygen atmosphere is performed in a temperature of above 90° C. In another embodiment, this reaction carried under air atmosphere or oxygen atmosphere is performed in a temperature of between 50 to 100° C. In another embodiment, this reaction carried under air atmosphere or oxygen atmosphere is performed in a temperature of between 60 to 90° C.

The compounds disclosed herein, as well as analogs of such compounds that will be readily apparent to those of ordinary skill in the art of medicinal chemistry upon consideration of this disclosure, can be prepared in a number ways using techniques well known to those of ordinary skill in the art. Exemplary methods for making particular compounds are described below. It is understood by those of ordinary skill in the art of organic synthesis that these methods are generalizable to the synthesis of compounds not explicitly described below upon consideration of the functionality of the molecule in view of the reagents and reactions disclosed. In view of the disclosed conditions, a person of ordinary skill in the art will recognize alternate methods for preparing analogous compounds that may have functional groups that are incompatible with the specific conditions disclosed herein.

In some embodiments, depending upon the functional groups present in a given compound, protecting groups for various groups may be preferred for masking the group during the transformation. Suitable protecting groups for various functionalities are described in Greene and Wuts Protective Groups in Organic Synthesis; 3rd Ed.; John Wiley & Sons, New York (1999).

In another embodiment, the present invention provides an iron based Cross Dehydrogenative Coupling chemistry synthetic path for the compounds disclosed herein. In another embodiment, the present invention provides iron catalyzed coupling reaction of ethyl 2-(2-methoxybenzoyl)acetate derivatives (compounds 2b and 2c see FIG. 1) with a variety of phenols a diversity-oriented synthesis of Coumestrol and its derivatives.

In another embodiment, the present invention provides a two-step retro-synthetic analysis of the coumestans is illustrated in FIG. 1. In another embodiment, a compound having a coumestan structure motif (1, 8a-8m, FIG. 1) is synthesized from the corresponding benzofurans 7a-7i by sequential demethylation and lactonization steps, while the latter is prepared using iron catalyzed oxidative cross coupling reactions between ethyl 2-(2-methoxybenzoyl)acetate derivatives 2b and 2c and the appropriate phenols (3a-3j) (FIG. 1).

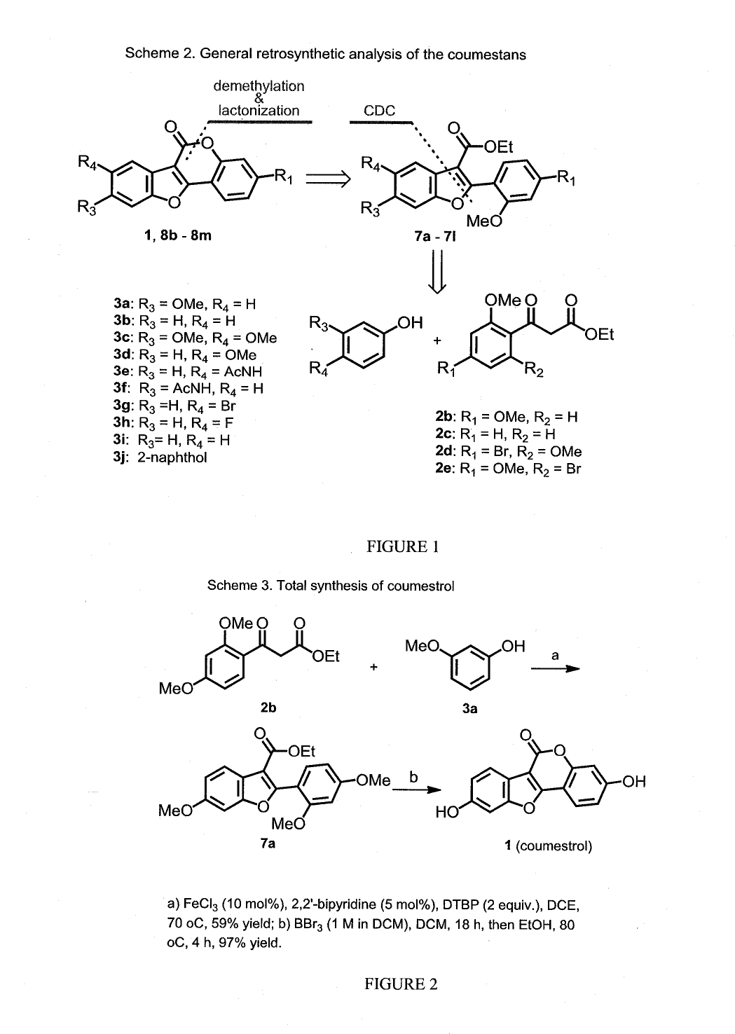

In another embodiment, the present invention provides that synthesis of coumestrol (see structure 1 in FIG. 2) an related compounds disclosed herein begin with the cross dehydrogenative coupling reaction between ethyl 2-(2,4-dimethoxybenzoyl)acetate (see structure 2b in FIG. 2, 1 equiv) and 3-methoxyphenol (see structure 3a in FIG. 2, 1.1 equiv), using FeCl₃ (1-20 mol %), 2,2′-bipyridine (1-19 mol %) or phenanthroline (1-19 mol %) as additive, and DTBP (1-10 equiv.) as the oxidant in DCE (0.05-2 M) at 50-80° C. for 1-10 h or alternatively, using FeCl₃ or FeCl₃(H₂O)₆ (1-20 mol %) under air or O₂ atmosphere. In another embodiment, under these conditions benzofuran (see structure 7a in FIG. 2) was obtained in 40-80% yield. In another embodiment, the conversion of the latter into structure 1 (FIG. 2) was carried out using a one-pot protocol: first, removal of the protecting groups such as but not limited to methyl groups which afforded the deprotected benzofuran intermediate; second, by switching to heated (above 40° C.) organic solvent such as but not limited to ethanol. In another embodiment, the lactonization step was accomplished and the resulting insoluble solid was filtered to afford coumestrol (structure 1 in FIG. 2) in over 90% yield.

In another embodiment, this synthesis protocol is applied to the formulas of the invention such as but not limited to coumestan (compound 8b) and 8-hydroxycoumestrol (compound 8c) (entries 1 and 2, Table 1). In another embodiment, the coupling reaction between ethyl 2-(2-methoxybenzoyl)acetate (2c) and phenol (compound 3b) afforded benzofuran (compound 7b), which was converted to coumestan, and 8c was synthesized starting from β-ketoester (compound 2b) and 3,4-dimethoxyphenol (compound 3c). In another embodiment, ethyl 2-benzoylacetates having ortho-methoxy group, such as (compounds 2a and 2b), reacted well and can be applied to members of the coumestan family.

In another embodiment, unnatural coumestrol analogues suitable for structure activity relationship study are also synthesized according to the process of the invention. In another embodiment, the presented synthesis path allows for the design and synthesis of novel ER ligands based on coumestrol and for the first time enables a comprehensive medicinal-chemistry study, with the flexibility to install substituents in almost all aromatic positions. In another embodiment, the hydrophobic ligand binding domain of ERs imposes an absolute structure requirement on effective binding to contain a nonpolar planar ring group having hydroxyl group(s) with a specific orientation.

In another embodiment, compounds such as but not limited to β-ketoester (compounds 2b and 2c) were used as coupling partners and were reacted with a variety of phenol derivatives (Table 1). In another embodiment, the oxidative coupling reaction of compound 2b with phenols bearing meta- and para-electron neutral and rich substituents (compounds 2a-2f) resulted in the formation of benzofurans of compounds such as 7a-7h (FIG. 2 and entries 2-7, Table 1). In another embodiment, electron deficient phenols, such as phenols of compounds 3g-3i, bearing p-Br, p-F and p-CF₃ groups, were used as coupling partners for synthesizing compounds such as 7i-7k.

In another embodiment, the conversion of benzofurans (such as compounds 7b-7j and 7m) to the corresponding coumestrol analogues was performed using BBr₃, BCl₃, TMSI, Pyridine hydrochloride, and other methods for demethylation that were described in Greene and Wuts Protective Groups in Organic Synthesis; 3rd Ed.; John Wiley & Sons, New York (1999).

In another embodiment, 3-ethoxycarbonylcoumestrol derivative (compound 8k) is obtained by converting benzofuran (compound 7k) bearing the trifluoromethyl group via acid-catalyzed alcoholysis of the acid-sensitive CF₃ group. In another embodiment, 3-trifluoromethylcoumestrol (compound 8l) is obtained by deprotecting a compound such as compound 7k (for example with BBr₃), and then refluxing under basic conditions (for example catalytic amount of triethylamine) in a hydrocarbon solvent, such as toluene

TABLE 1
Synthesis of coumestans via direct coupling of beta-ketoesters (2) and phenols
(compound 3) mediated by FeCl₃/2,2′-bipyridine/DTBP system

| entry | beta-ketoester (2) | phenols (3) | benzofuran (7) | coumestan (8) |
|---|---|---|---|---|
| 1 | 2c | 3b | 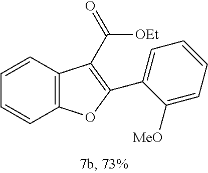 [SMILES: CCOC(=O)c1c(-c2ccccc2OC)oc2ccccc12] | 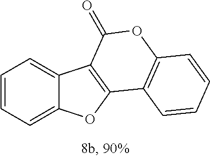 [SMILES: O=c1oc2ccccc2c2oc3ccccc3c12] |
| 2 | 2b | 3c | 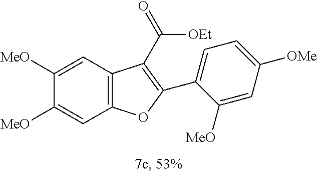 [SMILES: CCOC(=O)c1c(-c2ccc(OC)cc2OC)oc2cc(OC)c(OC)cc12] | 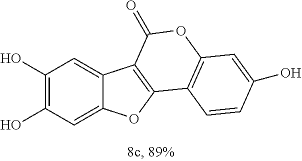 [SMILES: O=c1oc2cc(O)ccc2c2oc3cc(O)c(O)cc3c12] |
| 3 | 2b | 3b | 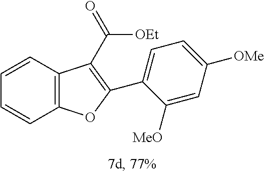 [SMILES: CCOC(=O)c1c(-c2ccc(OC)cc2OC)oc2ccccc12] | 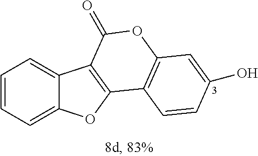 [SMILES: O=c1oc2cc(O)ccc2c2oc3ccccc3c12] |
| 4 | 2c | 3a | 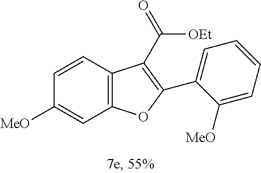 [SMILES: CCOC(=O)c1c(-c2ccccc2OC)oc2cc(OC)ccc12] | 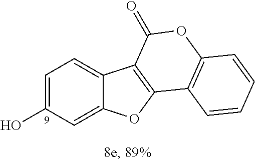 [SMILES: O=c1oc2ccccc2c2oc3cc(O)ccc3c12] |
| 5 | 2b | 3d | 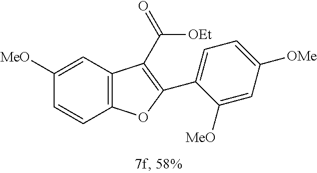 [SMILES: CCOC(=O)c1c(-c2ccc(OC)cc2OC)oc2ccc(OC)cc12] | 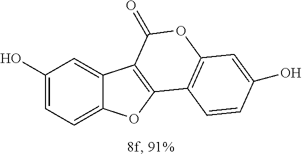 [SMILES: O=c1oc2cc(O)ccc2c2oc3ccc(O)cc3c12] |
| 6 | 2b | 3e | 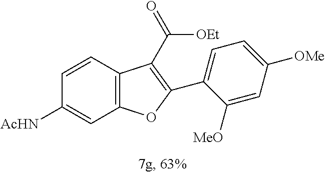 [SMILES: CCOC(=O)c1c(-c2ccc(OC)cc2OC)oc2cc(NC(C)=O)ccc12] | 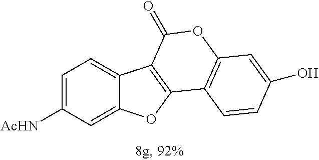 [SMILES: CC(=O)Nc1ccc2c(c1)oc1c3ccc(O)cc3oc(=O)c21] |
| 7 | 2b | 3f | 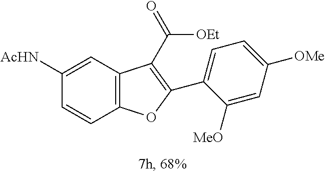 [SMILES: CCOC(=O)c1c(-c2ccc(OC)cc2OC)oc2ccc(NC(C)=O)cc12] | 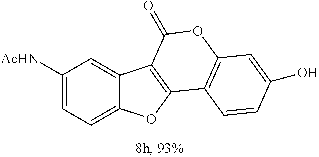 [SMILES: CC(=O)Nc1ccc2oc3c4ccc(O)cc4oc(=O)c3c2c1] |
| 8 | 2b | 3g | 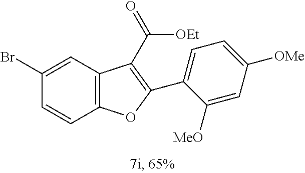 [SMILES: CCOC(=O)c1c(-c2ccc(OC)cc2OC)oc2ccc(Br)cc12] | 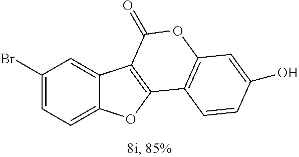 [SMILES: O=c1oc2cc(O)ccc2c2oc3ccc(Br)cc3c12] |
| 9 | 2b | 3h | 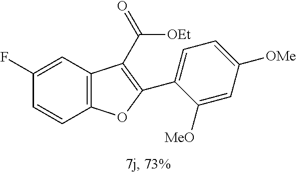 [SMILES: CCOC(=O)c1c(-c2ccc(OC)cc2OC)oc2ccc(F)cc12] | 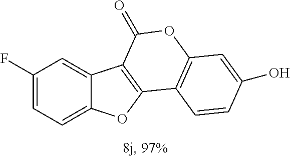 [SMILES: O=c1oc2cc(O)ccc2c2oc3ccc(F)cc3c12] |
| 10 | 2b | 3i | 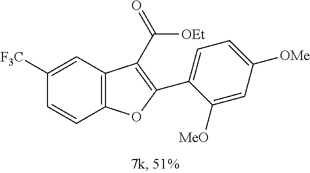 [SMILES: CCOC(=O)c1c(-c2ccc(OC)cc2OC)oc2ccc(C(F)(F)F)cc12] | 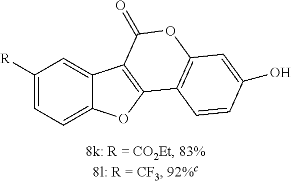 |
| 11 | 2b | 3j | 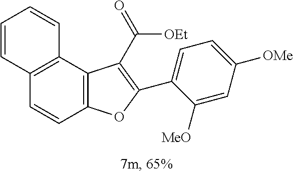 [SMILES: CCOC(=O)c1c(-c2ccc(OC)cc2OC)oc2ccc3ccccc3c12] | 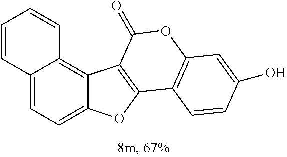 [SMILES: O=c1oc2cc(O)ccc2c2oc3ccc4ccccc4c3c12] |

In another embodiment, any compound as synthesized or disclosed herein is a compound of the invention that can be further utilized according to the methods of the invention. In another embodiment, provided here a synthesis path based on cross dehydrogenative coupling reaction of phenols and β-ketoseters for the preparation of a library of coumestrols. In another embodiment, provided here a synthesis path based on cross dehydrogenative coupling reaction of phenols and β-ketoseters for the preparation of a library of coumestrol SERMs. In another embodiment, this diversity-oriented synthesis allowed for structure activity relationship (SAR) study of the compounds described herein including natural products.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

General Procedures.

All reagents were of reagent grade quality, purchased commercially from Sigma-Aldrich, Alfa-Aesar, or Fluka, and used without further purification. Purification by column chromatography was performed on Merck chromatographic silica gel (40-60 μm). TLC analyses were performed using Merck silica gel glass plates 60 F₂₅₄.NMR spectra were recorded on Bruker DPX400, or DMX500 instruments; chemical shifts, given in ppm, are relative to Me₄Si as the internal standard or to the residual solvent peak. HR-MS data were obtained using a Thermoscientific LTQU XL Orbitrap HRMS equipped with APCI (atmospheric-pressure chemical ionization). Gas chromatography data were obtained using an Agilent 7820A GC equipped with FID detector working under standard conditions and an Agilent HP-5 column. HPLC analysis was carried out on an Agilent 1260 instrument equipped with a G4212-60008 photodiode array detector and a Agilent reverse phase ZORBAX Eclipse plus C18 3.5 μm column (4.6×100 mm). IR spectra were recorded on a Nicolet 380 FTIR spectrometer.

### Example 1

### First Novel Synthesis Path for Coumestrol Derivatives

This invention discloses a novel application for iron based CDC chemistry in the context of natural product synthesis. Based on the iron catalyzed coupling reaction of ethyl 2-(2-methoxybenzoyl)acetate derivatives (compounds 2b and 2c, FIG. 1) with a variety of phenols a diversity-oriented synthesis of coumestrol derivatives was developed (including a gram scale total synthesis of Coumestrol). In addition, the estrogenicity of the prepared analogues was evaluated by testing their effects on the proliferation of the estrogen receptor (ER)-dependent MCF-7 and of the ER-independent MDA-MB-231 breast cancer cell lines.

These SAR studies probed new SERMs such as but not limited to compound 8h (see Table 1) with potent ER dependent anticancer activity at the nanomolar scale. Some of these new compounds represent a novel type of ER modulators having acetamide group instead of hydroxy group.

The synthetic work in this project was commenced by developing an efficient entry to the coumestan family. The two-step retrosynthetic analysis of the coumestans is illustrated in FIG. 1. The coumestan structure motif (compounds 1, 8a-8m, Table 1) was synthesized from the corresponding benzofurans of compounds 7a-7i (Table 1) by sequential demethylation and lactonization steps. The latter was also prepared using iron catalyzed oxidative cross coupling reactions between ethyl 2-(2-methoxybenzoyl)acetate derivatives (compounds 2b and 2c, FIG. 1) and the appropriate phenols (compounds 3a-3j, FIG. 1).

The two steps total synthesis of coumestrol (compound 1) begin with the cross dehydrogenative coupling reaction between ethyl 2-(2,4-dimethoxybenzoyl)acetate (compound 2b, 1 equivalent) and 3-methoxyphenol (compound 3a, 1.1 equivalent), both commercially available, using FeCl₃ (10 mol %), 2,2′-bipyridine (5 mol %) or phenanthroline (5 mol %) as additive, and DTBP (2.5 equivalents) as the oxidant in DCE (0.5 M) at 70° C. for 8 hours (h). Serendipitously, under these conditions benzofuran (compound 7a, table 1) was obtained in 59% yield. The conversion of the latter into compound 1 was carried out using a one-pot protocol: First, removal of the methyl groups (BBr₃, 6 equiv, DCM, rt, overnight) afforded the deprotected benzofuran intermediate; then, by switching the solvent to boiling ethanol, the lactonization step was accomplished and the resulting insoluble yellowish solid was filtered to afford coumestrol (compound 1) in 97% yield. To demonstrate the possibility of scaling up this method for mass production, a gram scale (10 mmol scale) synthesis of coumestrol was successfully accomplished; over 1.6 g of the natural product was prepared in 59% overall yield.

After solving the production problem of coumestrol, the synthesis protocol was applied to other members of the coumestan family. Namely, coumestan (compound 8b, table 1) and 8-hydroxycoumestrol (compound 8c, table 1) (entries 1 and 2, Table 1). Thus, the coupling reaction between ethyl 2-(2-methoxybenzoyl)acetate (compound 2c) and phenol (compound 3b) afforded benzofuran (compound 7b) (73% yield), which was converted to coumestan in 90% yield, and compound 8c was synthesized starting from β-ketoester (compound 2b) and 3,4-dimethoxyphenol (compound 3c) in 52% yield for the two steps. The latter could be converted to the Medicagol natural product in only one synthetic step. While ethyl 2-benzoylacetates having ortho-methoxy group, such as compound 2a and compound 2b, reacted well and can be applied to many members of the coumestan family, the repeated attempts to react ethyl 2-benzoylacetates having two ortho-substituents such as ethyl 2-(4-bromo-2,6-dimethoxybenzoyl)acetate (compound 2d) and ethyl 2-(6-bromo-2,4-dimethoxybenzoyl)acetate (compound 2e), which upon successful coupling could provide an entry to the wedelolactone natural product, failed to react.

Encouraged by the success of the present syntheses, the synthesis of unnatural coumestrol analogues suitable for structure activity relationship study, were further conducted. The presented method allows for the design and synthesis of novel ER ligands based on coumestrol and for the first time enables a comprehensive medicinal-chemistry study, with the flexibility of installing substituents in almost all aromatic positions. The hydrophobic ligand binding domain of ERs imposes an absolute structure requirement on effective binding to contain a nonpolar planar ring group having hydroxyl group(s) with a specific orientation.

Based on the above findings, designing coumestrol derivatives having at least one phenol group installed (will direct the ligand in to the ligand-binding domain (vide infra)) was commenced.

Synthetically, β-ketoester (compounds 2b and 2c, FIG. 1, Table 1) were chosen as the coupling partners and were reacted with a variety of phenol derivatives (Table 1). The oxidative coupling reaction of compound 2b with phenols bearing meta- and para-electron neutral and rich substituents (compounds 2a-2f) resulted in the formation of benzofurans (compounds 7a-7h) in moderate yields (53%-68%, Scheme 3 and entries 2-7, Table 1). Although phenols bearing ortho-alkyl substituents were found to be suitable coupling partners, the reaction with 2-methoxyphenol gave a complex reaction mixture and the coupling product could only be detected in a disappointing amount (<10% yield). Electron deficient phenols, such as phenols (compounds 3g-3i, bearing p-Br, p-F and p-CF₃ groups), were found to be good partners as well, and benzofurans (compounds 7i-7k) have been isolated in 65%, 73% and 51% yields, respectively. Less activated phenols, such as 4-cyanophenol, 4-formylphenol or 4-(ethoxycarbonyl)phenol failed to react under our general conditions.

The conversion of benzofurans (compounds 7b-7j and 7m, table 1) to the corresponding coumestrol analogues was performed in good to excellent yields using BBr₃ (DCM, then boiling ethanol). However, initial attempts to convert benzofuran (compound 7k) bearing the trifluoromethyl group resulted in formation of the 3-ethoxycarbonylcoumestrol derivative (compound 8k) in 84% yield, as a result of acid-catalyzed alcoholysis of the acid-sensitive CF₃ group. Alternatively, when compound 7k was deprotected first with BBr₃, and then refluxed in toluene in the presence of a catalytic amount of triethylamine (50 mol %) for 30 min, the desired 3-trifluoromethylcoumestrol (compound 8l) was isolated after column chromatography in 92% yield; previous attempts to prepare —CF₃ substituted coumestrol using different synthetic approaches failed.

In parallel to the synthetic efforts the structural motifs that responsible of the estrogenic activity of compound 1 were also studied. For this purpose an approach combining molecular modeling techniques with a molecular biology study, was taken. Specifically, the effect of coumestans on the proliferation of breast cancer cell lines was studied.

### Activity

Cell Lines:

MCF-7 cells and MDA-MB-231 cells were maintained in Costar T75 flasks with Dulbeccos Modified Eagle Medium (DMEM) supplemented with 2 mM glutamine and 10% fetal bovine serum (Biological Industries Beit Haemek, LTD).

To deplete cells of estrogens, they were passaged for 1 week in phenol red-free DMEM supplemented with 10% estrogen-depleted calf serum (DCS/MEM) which was made by sequential treatment of calf serum with sulfatase and dextran-coated charcoal (Biological Industries Beit Haemek, LTD).

Proliferation Studies:

MCF-7 and MDA-MB-231 cells were plated in 96 well dishes (Costar) at approximately 5,000 cells/well and 2500 cells/well respectively in 100 ul medium. One day after plating (Day 0) 100 μl of the treatment media were added.

Final volume in each well was 200 μl. Each chemical was diluted to a final concentration of 10⁻³M. At day 0 compounds 1 and 8b-8m (table 1) were diluted aside and 100 μl from each dilution were added to each well. The following dilution steps were performed: 2×10⁻⁶M (4 μl of 10⁻³M in 2 mL), 2×10⁻⁷ M (200 μl of 2×10⁻⁶M in 2 mL), 2×10⁻⁸M (200 μl of 2×10⁻⁷M in 2 mL) and 2×10⁻⁹M (200 μl of 2×10⁻⁸M in 2 mL).

Cell proliferation was quantified by colorimetric MTT assay. The use and validity of the MTT assay in MCF-7 cells is described by Martikainen et al.

Measurement of cell viability and proliferation were based on the reduction of tetrazolium salts using the MTT kit (Biological Industries Beit Haemek, LTD) according to the manufacturer instructions. The yellow tetrazolium MTT (3-(4,5-dimethylthiazolyl-2)-2,5-diphenyltetrazolium bromide) is reduced by metabolically active cells, in part by the action of dehydrogenase enzymes, to generate reducing equivalents such as NADH and NADPH. The resulting intracellular purple formazan can be solubilized and quantified by spectrophotometric means. The MTT Reagent yields low background absorbance values in the absence of cells. MTT assay was used to evaluate cell number in each well.

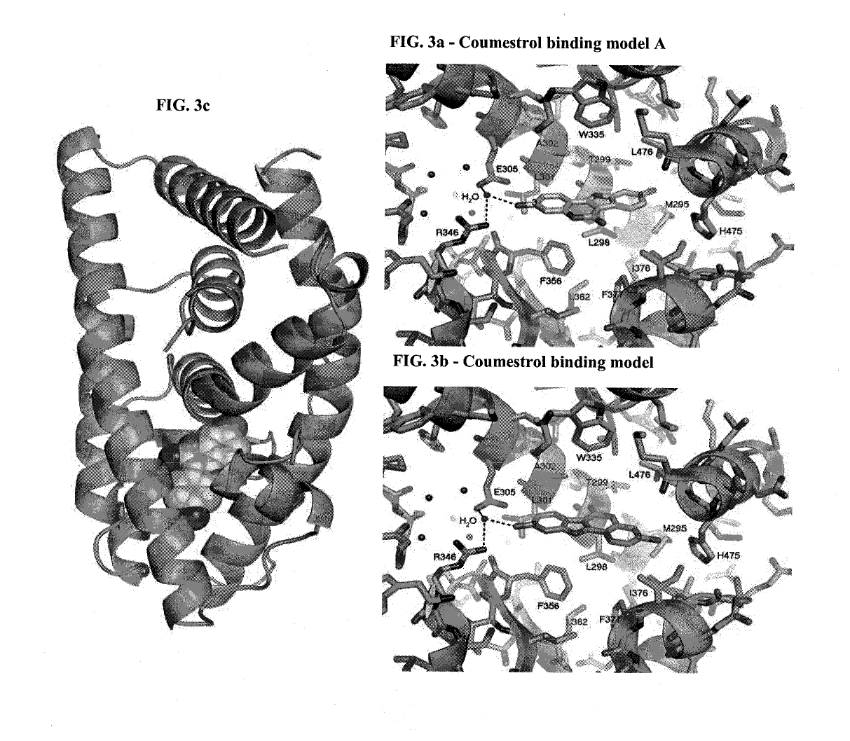

The dimensions of the ERs binding sites, as reflected from many solved crystal structures, suggest that coumestrol recognition can be achieved inside the hydrophobic pocket in two opposite binding modes, as presented in FIG. 3; either the 3-hydroxy group interact through hydrogen bonding with a buried water molecule in the structurally conserved polar pocket form by Glu₃₀₅ and Arg₃₄₆ residues (binding model A, FIG. 3a), or alternatively, the 9-hydroxy group is pointed toward that polar pocket as illustrate in binding model B (FIG. 3b). In both cases, several hydrophobic interactions of surrounding hydrophobic amino acids (such as Leu₂₉₈ and Phe₃₅₆) restrict the conformational freedom of the ligand. Finally, the remained hydroxyl group can bind at the end of the cavity with the flexible His₄₇₅ residue.

The two different binding models represent inverted conformational arrangement of compound 1 in the hydrophobic pocket. Although an X-ray co-crystal structure of coumestrol complex to ERα or ERβ can provide the needed evidence to coumestrol's preferred binding form, such a crystal is missing. Despite that, the difference in pKa values of the two hydroxyl groups (7.5 and 9.1, to the 3- and 9-hydroxyl respectively) and the structures of co-crystals of the proteins (ERα and ERβ) with resemble ligands and co-crystal of coumestrol with other enzyme suggest that the conformation in which the 3-hydroxy group is interact to the Glu₃₀₅ and Arg₃₄₆ residues is more significant. In order to determine which of the two phenol groups have stronger impact on the estrogenic activity of coumestrol, 3-hydroxycoumestn (compound 8d) and 9-hydroxycoumestan (compound 8e), were prepared and the proliferative impact of the two isomers on ER-positive breast cancer line, MCF-7 was recorded. While compound 8e was found to have moderate activity with IC₅₀ value of 0.56 μM (Table 2, entry 4), the other isomer, compound, 8d did not showed any proliferative effect on the MCF-7 cells (entry 3). These results are in agreement with the assumption that the strong interaction between the Glu₃₅₃ and Arg₃₉₄ residues in the ER binding site takes place with the 3-hydroxy group of coumestrol. Therefore, in terms of SAR the 9-hydroxy group can be removed and replaced with different substituents.

TABLE 2
IC₅₀ values (compounds are provided in table 1)

| entry | compound | IC₅₀ (10⁻⁹ M) |
|---|---|---|
| 1 | 1 | 73 |
| 2 | 8b | NA |
| 3 | 8c | NA |
| 4 | 8d | 568 |
| 5 | 8e | NA |
| 6 | 8f | NA |
| 7 | 8g | 30 |
| 8 | 8h | 9 |
| 9 | 8i | 107 |
| 10 | 8j | 220 |
| 11 | 8k | 58 |
| 12 | 8l | 170 |
| 13 | 8m | 180 |

^{b}NA = Not Active

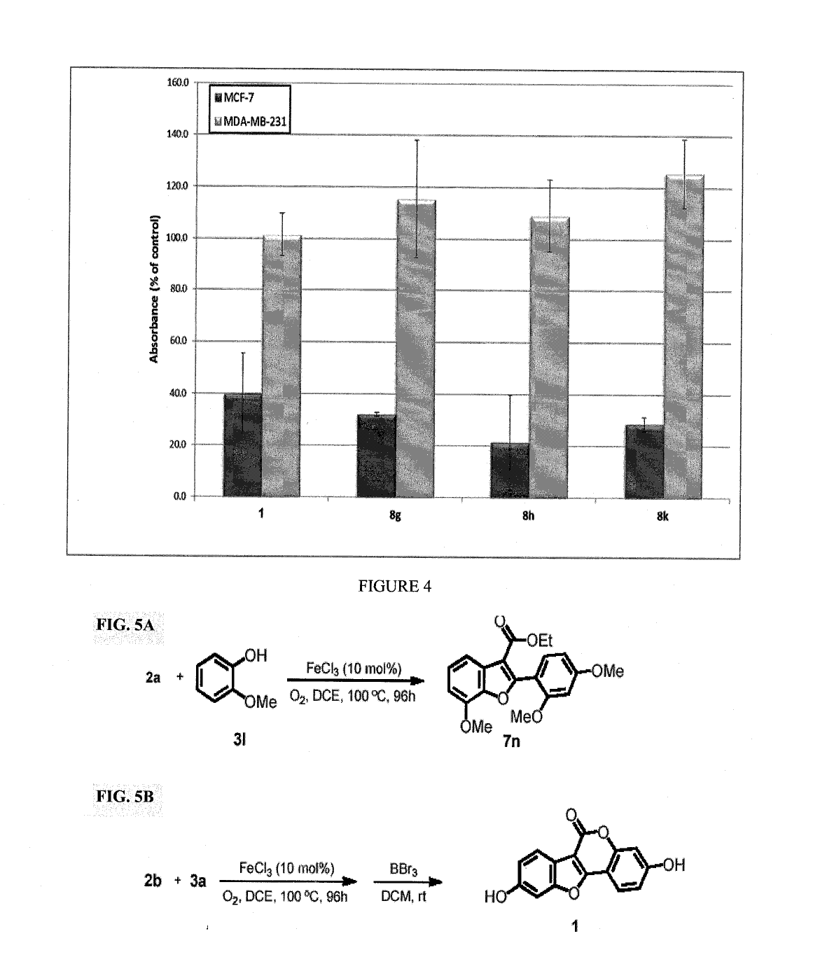

Based on these findings a small library of coumestrol derivatives was prepared having different substituents at the C-8 and C-9 positions. The cell proliferation effect on the MCF-7 cell line (estrogen dependent cells) was recorded for all coumestan derivatives (see IC₅₀ values in Table 2). In order to determine that the proliferation effect observed in the estrogen-dependent MCF-7 breast cancer cells involved binding of the coumestan derivatives to the estrogen receptor, the effect of these compounds on estrogen-independent MDA-MB-321 breast cancer cells was also tested (FIG. 4). Not surprisingly, all tested compounds were found inactive and did not block the proliferation of these cells, supporting the assumption that the synthetic compounds target the estrogen receptor.

The superior estrogenic activity of coumestrol over other members of the coumestan family is in consistent with our results that compounds 8b, 8c, 8d, 8e and 8f (see table 1) having different oxygenation pattern than coumestrol are at least one order of magnitude less active than natural compound 1.

Moderate estrogenic activity was obtained when the benzofuran ring was substituted with bulky groups such as 8-Br (compound 8i, table 1), 8-CF₃ (compound 8l, table 1) or fused ring as in napthocoumestrol (compound 8m), having IC₅₀ values of 107, 170 and 180 nM respectively. The replacement of the 9-hydroxy group of coumestrol with 8-CO₂Et (compound 8k, table 1) or 9-AcNH (compound 8g, table 1) groups influenced dramatically on the estrogenic activity (IC₅₀ values of 58 and 30 nM respectively). Docking of the latter compound into the ligand binding domain of ERβ suggesting that the NH group is located in a right orientation to form hydrogen bond with the His₄₇₅ residue. In addition, hydrophobic interactions took place between the acetamide group of (compound 8g) with close hydrophobic amino acid residues such as Leu₄₇₆ (˜2.5 Å distance), Met₄₇₉ (˜2.8 Å), Met₂₉₅ (˜3.1 Å) and Thr₂₉₉ (˜3.4 Å).

Next, the impact of the location of the acetamide group on the estrogenic activity was examined. For this purpose 8-acetamidecoumestrol (compound 8h) was prepared and tested against MCF-7 breast cancer cells. Fortunately, this tactic paid off as the latter compound was found to have potent activity against these cells with IC50 value of <1 nM. An examination of the latter compound in the ERβ ligand binding domain showed poor compatibility at the end of the cavity, suggesting that binding of compound 8h in the ER should result with conformational change of the flexible His₄₇₅ moiety that will influence the overall structure of the receptor.

In conclusion, replacement of the hydroxyl of a SERM with amide group was never reported. The synthesis reported herein is based on cross dehydrogenative coupling reaction of phenols and β-ketoseters and was successfully applied for the preparation of a library of coumestrol SERMs. This diversity-oriented synthesis allowed for the first time to perform structure activity relationship study of the important natural product. These studies revealed that the 3-hydroxy group in coumestrol is crucial for the activity whereas the 9-hydroxy group can be replaced. Indeed, when acetamide group was introduced (compounds 8g and 8h) the cell-proliferation effect was intensified.

### Example 2

### First Novel Non-Toxic Synthesis Path for Coumestrol Derivatives

Although, the coupling of beta-ketoesters 2 and phenols 3 (as in example 1) is providing an easy access to a variety of coumestrol derivatives, the reaction requires the use of hazardous materials—such as DTBP as the oxidant.

The NHPI/O₂ oxidation system was assumed to be a good solution for safety concerns, but also because it allows for more environmentally friendly and economical reactions, and in the case of phenol coupling reactions it should eliminate the Friedel-Crafts alkylation side reaction resulted from the utilization of DTBP and TBHP in the reactions.

In these experiments, ethyl 2-(2,4-dimethoxybenzoyl)acetate (compound 2b, 1 equiv) and 3-methoxyphenol (compound 3a, 1.1 equiv) were mixed in DCE at 100° C. in the presence of FeCl₃ (10 mol %) and NHPI (5 mol %) under oxygen atmosphere, the reaction went to completion within 24 h affording coupling product 7a in 61% isolated yield (Table 3, entry 1). Increasing the amount of NHPI to 20 mol % had negative effect on the yield (53%, Table 3 entry 2).

TABLE 3
Optimization of the CDC reaction of β-ketoester 2b and phenol 3b
under oxygen and aerobic conditions.^{a}

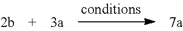

| | | | Time | yield^{b} |
|---|---|---|---|---|
| entry | conditions | solvent | (h) | (%) |
| 1 | FeCl₃ (10 mol %), NHPI (5 mol %), | DCE | 24 | 61 |
| | O₂ balloon | | | |
| 2 | FeCl₃ (10 mol %), NHPI (20 mol %), | DCE | 24 | 53 |
| | O₂ balloon | | | |
| 3 | FeCl₃ (10 mol %), O₂ balloon | DCE | 24 | 63 |
| 4 | FeCl₃ (10 mol %), 2,2′-bipyridine (5 mol %), | DCE | 24 | [26]^{c} |
| | O₂ balloon | | | |
| 5 | FeCl₃ (10 mol %), atmospheric air | DCE | 48 | 52 |
| 6 | FeCl₃ · (H₂O)₆ (10 mol %), O₂ balloon | PhMe | 9 | 50 |

^{a}All reaction were carried out with compound 2b (0.5 mmol), compound 3a (0.65 mmol) in DCE (0.25 M) at 100° C.
^{b}Isolated yields.
^{c}NMR yields are given in square brackets; 1,3,5-trimethoxy benzene was used as internal standard; NHPI = N-hydroxyphthalimide, DCE = 1,2-dichloroethane

Furthermore, when the reaction was performed in the absence of NHPI, benzofuran of compound 7a was isolated in moderate 63% yield (Table 3, entry 3); indicating that NHPI is not playing a role in the reaction mechanism. The addition of 2,2′-bipyridine (5 mol %) to the reaction mixture slowed down the process and after 24 h only partial conversion was observed (Table 3, entry 4). To simplify the method even further, the reaction was performed under air. Although, the reaction is slower and requires longer reaction time (48 h) the desired coupling product of compound 7a was isolated in 53% yield. Finally, when the reaction was performed in toluene as a solvent shorter the reaction was completed within 9 hours (h) affording the desired product in 58% yield (entry 6).

### Example 3

### Synthesis of Ethyl 2-(2,4-Dimethoxyphenyl)-6-Methoxybenzofuran-3-Carboxylate (7A)

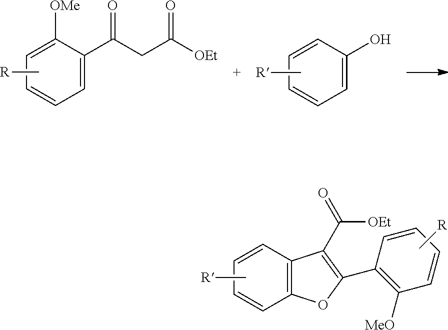

Method A:

Di-tert-butyl peroxide (1.7 ml, 19.8 mmol, 2.5 equiv) was added drop-wise into a stirred solution of ethyl 3-(2,4-dimethoxyphenyl)-3-oxopropanoate (2 g, 7.94 mmol, 1 equiv) and 3-methoxy phenol (1.08 g, 8.73 mmol, 1 equiv), 2,2′-bipyridine (0.062 g, 0.4 mmol, 0.05 equiv) and FeCl₃ (0.13 g, 0.8 mmol, 0.1 equiv) in 1,2-dichloroethane (0.5 M) under nitrogen atmosphere at room temperature. The reaction mixture was heated to 70^{˜}C for 8 hours, cooled to room temperature, quenched with saturated NaHCO₃ (10 mL) and extracted with EtOAc (3×10 mL). The combined organic layer was washed with saturated NaHCO₃ (10 mL), water (10 mL) and dried over Na₂SO₄. The solvent was removed under reduced pressure and the residue was purified by flash column chromatography over silica gel (ethyl acetate-hexanes, 2:8) affording compound 7a (1.72 g, 61%) as a colorless solid. ¹H NMR (400 MHz, CDCl₃, ppm) δ 7.88 (d, J=8.6 Hz, 1H), 7.48 (d, J=8.5 Hz, 1H), 7.04 (d, J=2.2 Hz, 1H), 6.96 (dd, J=8.6, 2.2 Hz, 1H), 6.59 (dd, J=8.5, 2.2 Hz, 1H), 6.54 (d, J=2.2 Hz, 1H), 4.3 (q, J=7.1 Hz, 2H), 3.86 (s, 3H), 3.85 (s, 3H), 3.79 (s, 3H), 1.29 (t, J=7.1 Hz, 3H); ¹³C NMR (100 MHz, CDCl₃, ppm) δ 164.0, 162.4, 158.9, 158.1, 157.6, 154.9, 132.3, 122.0, 120.0, 112.5, 112.2, 110.3, 104.3, 98.6, 95.6, 60.1, 55.6, 55.5, 55.4, 14.2; IR (KBr): 1700.9, 1623.8, 1500.4 cm-1; HRMS (ESI): m/z calcd for C₂₀H₂₁O₆ [M+H]⁺ 357.1332. found 357.1323.

Alternatively:

A solution of ethyl 3-oxo-3-arylpropanoate (1.0 equiv), phenol (1.3 equiv), and FeCl₃ (0.1 equiv) in DCE (0.5 M) were heated to 100° C. under O₂ atmosphere (O₂ balloon). After completion, the reaction was quenched with saturated NaHCO₃ (10 mL) and extracted with EtOAc (3×10 mL). The combined organic layer was washed with saturated NaHCO₃ (10 mL), water (10 mL) and dried over Na₂SO₄. The solvent was removed under reduced pressure and the residue was purified over flash column chromatography on silica gel.

Alternatively:

Ethyl 3-(2,4-dimethoxyphenyl)-3-oxopropanoate (0.126 mg, 0.5 mmol), phenol (0.081 mg, 0.65 mmol), and FeCl₃ (8 mg, 0.05 mmol) in DCE (1 mL, 0.5 M) were heated to 100° C. under O₂ balloon for 24 h. The crude residue was purified (ethyl acetate-hexanes, 2:8) affording compound 7a (112 mg, 63%) as a colorless solid.

### Example 4

### Synthesis of Coumestrol (1)

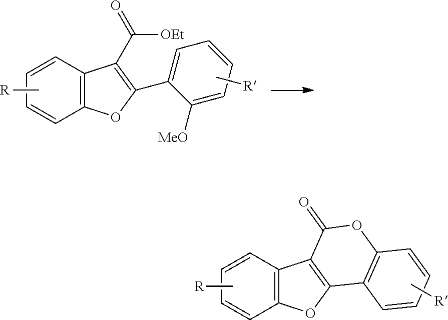

A solution of BBr₃ (1 M in DCM, 29 mL, 0.029 mol) was added drop-wise into a stirred solution of benzofuran of compound 7a (1.72 g, 4.83 mmol) in dry DCM (50 mL) at 0° C. under nitrogen atmosphere. The reaction mixture was allowed to warm to room temperature and further stirred overnight. After quenching the reaction with EtOH (1 ml) the volatiles were removed under reduced pressure and the residue was dissolved in EtOH (5 ml). The mixture was refluxed for 3 hours until TLC showed complete conversion and the desired product was filtered, washed with EtOH (1 ml) and dried under vacuum affording coumestrol (1.26 g, 97%) as a yellow solid. ¹H NMR (400 MHz, DMSO-d₆, ppm) δ 10.71 (s, 1H), 10.04 (s, 1H), 7.85 (d, J=8.1 Hz, 1H), 7.68 (d, J=8.5 Hz, 1H), 7.16 (d, J=2.0 Hz, 1H), 6.86-6.98 (m, 3H); ¹³C NMR (100 MHz, DMSO-d₆, ppm) δ 161.7, 160.1, 158.2, 157.5, 156.5, 155.2, 123.3, 121.2, 115.1, 114.6, 114.3, 104.7, 103.6, 102.6, 99.2.

One-Pot Synthesis of Coumestrol:

Ethyl 3-(2,4-dimethoxyphenyl)-3-oxopropanoate (2 g, 7.94 mmol), phenol (1.28 g, 10.3 mmol), and FeCl₃ (0.127 g, 0.79 mmol) in DCE (16 mL, 0.5 M) were heated to 100° C. under O₂ balloon for 84 h. The mixture was cooled to room temperature filtered through a plug of silica to remove metal residues and the volatiles were removed under reduced pressure and kept under high vacuum pump for 2 h. The crude mixture was dissolved in dry DCM (20 mL) and stirred at 0° C. under nitrogen atmosphere. A solution of BBr₃ (1 M in DCM, 32 mL) was added slowly via syringe and the mixture was stirred at room temperature for 24 h. Ethanol was added slowly (5 mL) and the volatiles were removed under reduced pressure. The remain crude was dissolved in a solution of ethanol-water (1:1, 30 mL) and refluxed for 3 hours until TLC showed completion of the reaction. The desired product was filtered, washed with EtOH (1 ml) and dried under vacuum affording coumestrol (1.8 g, 84% yield in 87% purity according to HPLC analysis). Pure coumestrol was obtained by purification (ethyl acetate: hexane, 8:2) over silica gel.

Coumestan (8b) (from table 1):

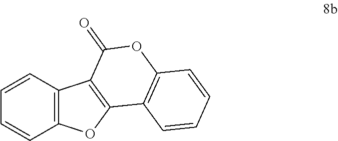 [SMILES: O=c1oc2ccccc2c2oc3ccccc3c12]

Ethyl 2-(2-methoxyphenyl)benzofuran-3-carboxylate (7b) (148 mg, 0.5 mmol) was treated with BBr₃ (1 mL, 1 mmol) according to general method C affording compound 8b (106 mg, 90%) as a white solid. ¹H NMR (400 MHz, CDCl₃, ppm) δ 8.06-8.15 (m, 1H), 7.99 (dd, J=7.7, 1.4 Hz, 1H), 7.61-7.66 (m, 1H), 7.58 (ddd, J=8.4, 7.0, 1.5 Hz, 1H), 7.34-7.49 (m, 4H); ¹³C NMR (100 MHz, CDCl₃, ppm) δ 159.9, 158.0, 155.4, 153.6, 131.9, 126.7, 125.2, 124.6, 123.4, 121.8 (2 carbons), 117.4, 112.5, 111.7, 105.8; IR (KBr): 1700.9, 1581.4 cm⁻¹; HRMS (ESI): m/z calcd for C₁₅H₉O₃ [M+H]⁺ 237.0546. found 237.0542.

Compound 8c (from table 1):

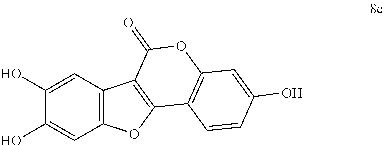 [SMILES: O=c1oc2cc(O)ccc2c2oc3cc(O)c(O)cc3c12]

ethyl 2-(2,4-dimethoxyphenyl)-5,6-dimethoxybenzofuran-3-carboxylate (7c) (193 mg, 0.5 mmol) was treated with BBr₃ (4 mL, 4 mmol) according to general method C affording compound 8c (126 mg, 89%) as a white solid. ¹H NMR (400 MHz, DMSO-d₆, ppm) δ 7.80 (d, J=8.5 Hz, 1H), 7.21 (s, 1H), 7.15 (s, 1H), 6.89 (dd, J=8.6, 1.9 Hz, 1H), 6.87 (d, J=1.9 Hz, 1H); ¹³C NMR (100 MHz, DMSO-d₆, ppm) δ 161.5, 159.7, 158.5, 155.0, 149.4, 146.2, 145.0, 123.2, 114.6, 114.3, 105.4, 105.0, 103.6, 102.8, 99.6; HRMS (ESI): m/z calcd for C₁₅H₈O₆ [M+H]⁺ 285.0393. found 285.0390.

Compound 8d (from table 1):

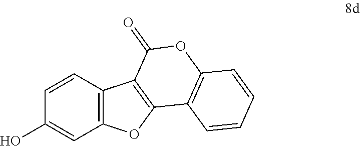 [SMILES: O=c1oc2ccccc2c2oc3cc(O)ccc3c12]

ethyl 6-methoxy-2-(2-methoxyphenyl)benzofuran-3-carboxylate (7d) (163 mg, 0.5 mmol) was treated with BBr₃ (2 mL, 2 mmol) according to general procedure C affording compound 8d (112 mg, 89%) as a white solid. ¹H NMR (400 MHz, DMSO-d₆, ppm) δ 10.13 (s, 1H), 7.96 (dd, J=7.8 and 1.4 Hz, 1H), 7.71 (d, J=8.3 Hz, 1H), 7.64 (ddd, J=8.4, 7.4 and 1.6 Hz, 1H), 7.54 (d, J=8.4 Hz, 1H), 7.42-7.47 (m, 1H), 7.17 (d, J=2.0 Hz, 1H), 6.96 (dd, J=8.4 and 2.0 Hz, 1H); ¹³C NMR (100 MHz, DMSO-d₆, ppm) δ 158.6, 158.1, 157.6, 156.8, 152.9, 132.0, 125.3, 121.7, 121.5, 117.4, 114.8, 114.7, 112.5, 105.8, 99.0; HRMS (ESI): m/z calcd for C₁₅H₈O₄ [M+H]⁺ 253.0495. found 253.0494.

Compound 8e (from table 1):

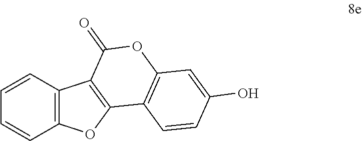 [SMILES: O=c1oc2cc(O)ccc2c2oc3ccccc3c12]

ethyl 2-(2,4-dimethoxyphenyl)benzofuran-3-carboxylate (7e) (163 mg, 0.5 mmol) was treated with BBr₃ (2 mL, 2 mmol) according to general procedure C affording compound 8e (105 mg, 83%) as a white solid. ¹H NMR (400 MHz, DMSO-d₆, ppm) δ 10.77 (s, 1H), 7.82-7.87 (m, 1H), 7.82 (d, J=8.6 Hz, 1H), 7.74-7-77 (m, 1H), 7.39-7.42 (m, 2H), 6.90 (dd, J=8.5 and 2.1 Hz, 1H), 6.86 (d, J=2.1 Hz, 1H); ¹³C NMR (100 MHz, DMSO-d₆, ppm) δ 162.2, 160.9, 157.8, 155.6, 154.8, 126.5, 125.6, 123.6, 123.5, 120.7, 114.2, 112.3, 104.2, 103.4, 102.1; HRMS (ESI): m/z calcd for C₁₅H₈O₄ [M+H]⁺ 253.0495. found 253.0493.

Compound 8f (from table 1):

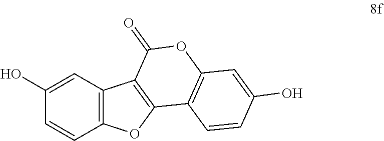 [SMILES: O=c1oc2cc(O)ccc2c2oc3ccc(O)cc3c12]

(table 1) Ethyl 2-(2,4-dimethoxyphenyl)-5-methoxybenzofuran-3-carboxylate (7f) (178 mg, 0.5 mmol) was treated with BBr₃ (3 mL, 3 mmol) according to general method C affording compound 8f (121 mg, 91%) as a white solid. ¹H NMR (400 MHz, DMSO, ppm) δ 10.75 (s, 1H), 9.65 (s, 1H), 7.81 (d, J=8.7 Hz, 1H), 7.57 (d, J=8.9 Hz, 1H), 7.22 (d, J=2.4 Hz, 1H), 6.90 (dd, J=8.6, 2.2 Hz, 1H), 6.87 (d, J=2.2 Hz, 1H), 6.85 (dd, J=8.5, 2.5 Hz, 1H); ¹³C NMR (100 MHz, DMSO-d₆, ppm) δ 162.1, 161.3, 158.0, 155.6, 155.5, 148.8, 124.4, 123.5, 114.7, 114.2, 112.8, 105.6, 104.4, 103.5, 102.2; IR (KBr): 3270.8, 1724.1, 1600.4 cm-1; HRMS (ESI): m/z calcd for C₁₅H₉O₅ [M+H]⁺ 269.0444. found 269.0447.

Compound 8g (from table 1):

ethyl 6-acetamido-2-(2,4-dimethoxyphenyl)benzofuran-3-carboxylate (7g) (192 mg, 0.5 mmol) was treated with BBr₃ (2 mL, 2 mmol) according to general method C affording compound 8g (142 mg, 92%) as a white solid. ¹H NMR (400 MHz, DMSO-d₆, ppm) δ 10.71 (br s, 1H), 10.21 (s, 1H), 8.19 (d, J=1.0 Hz, 1H), 7.79 (d, J=8.5 Hz, 1H), 7.69 (d, J=8.4 Hz, 1H), 7.39 (dd, J=8.4, 1.4 Hz, 1H), 6.88 (dd, J=8.6, 2.0 Hz, 1H), 6.83 (d, J=2.0 Hz, 1H), 2.06 (s, 3H); ¹³C NMR (100 MHz, DMSO-d₆, ppm) δ 169.0, 161.9, 160.6, 157.8, 155.3, 155.1, 138.4, 123.3, 120.5, 118.3, 116.9, 114.1, 104.3, 103.4, 102.4, 102.2, 24.5; IR (KBr): 3321.3, 1727.9, 1670.2, 1631.5 cm-1; HRMS (ESI): m/z calcd for C₁₇H₁₂NO₅ [M+H]⁺ 310.0710. found 310.0710.

Compound 8h (from table 1):

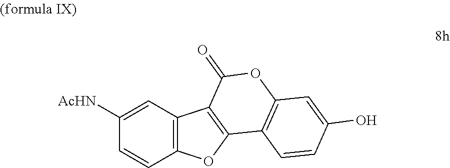 [SMILES: CC(=O)Nc1ccc2oc3c4ccc(O)cc4oc(=O)c3c2c1]

ethyl 5-acetamido-2-(2,4-dimethoxyphenyl)benzofuran-3-carboxylate (7h) (192 mg, 0.5 mmol) was treated with BBr₃ (2 mL, 2 mmol) according to general method C affording compound 8h (143 mg, 93%) as a white solid. ¹H NMR (400 MHz, DMSO-d₆, ppm) δ 10.84 (br s, 1H), 10.09 (s, 1H), 8.20 (d, J=2.0 Hz, 1H), 7.78 (d, J=8.6 Hz, 1H), 7.64 (d, J=8.9 Hz, 1H), 7.52 (dd, J=9.0, 2.1 Hz, 1H), 6.88 (dd, J=8.7, 2.1 Hz, 1H), 6.84 (d, J=2.1 Hz, 1H), 2.04 (s, 3H); ¹³C NMR (100 MHz, DMSO-d₆, ppm) δ 168.9, 162.3, 161.5, 158.0, 155.7, 150.9, 137.3, 123.8, 123.7, 118.1, 114.4, 112.3, 110.7, 104.4, 103.5, 102.3, 24.5; HRMS (ESI): m/z calcd for C₁₇H₁₂NO₅ [M+H]⁺ 310.0710. found 310.0709.

Compound 8i (from table 1):

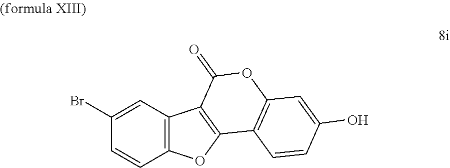 [SMILES: O=c1oc2cc(O)ccc2c2oc3ccc(Br)cc3c12]

ethyl 5-bromo-2-(2,4-dimethoxyphenyl)benzofuran-3-carboxylate (7i) (195 mg, 0.5 mmol) was treated with BBr₃ (2 mL, 2 mmol) according to general method C affording compound 8i (140 mg, 85%) as a white solid. ¹H NMR (400 MHz, DMSO-d₆, ppm) δ 10.9 (s, 1H), 7.83 (d, J=1.8 Hz, 1H), 7.79 (d, J=8.6 Hz, 1H), 7.70 (d, J=8.7 Hz, 1H), 7.53 (dd, J=8.8, 1.4 Hz, 1H), 6.87 (dd, J=8.6, 1.5 Hz, 1H) 6.82 (d, J=1.7 Hz, 1H); ¹³C NMR (100 MHz, DMSO-d₆, ppm) δ 162.9, 162.2, 157.8, 156.0, 154.0, 129.4, 125.9, 124.1, 123.0, 118.1, 114.7, 114.6, 104.1, 103.7, 101.6; HRMS (ESI): m/z calcd for C₁₅H₈BrO₄ [M+H]⁺ 330.9600. found 330.9603.

Compound 8j (from table 1):

ethyl 2-(2,4-dimethoxyphenyl)-5-fluorobenzofuran-3-carboxylate (7j) (172 mg, 0.5 mmol) was treated with BBr₃ (2 mL, 2 mmol) according to general method C affording compound 8j (131 mg, 97%) as a white solid. ¹H NMR (500 MHz, DMSO-d₆, ppm) δ 10.90 (s, 1H), 7.67 (d, J=8.6 Hz, 1H), 7.66 (dd, J=9.0, 4.0 Hz, 1H), 7.35 (dd, J=8.1, 2.7 Hz, 1H), 7.17 (ddd, J=9.0, 8.1, 2.7 Hz, 1H), 6.82 (dd, J=8.6, 2.2 Hz, 1H), 6.74 (d, J=2.1 Hz, 1H); ¹³C NMR (125 MHz, DMSO-d₆, ppm) δ 162.6, 162.2, 160.0 (d, ¹J_{CF}=240 Hz), 157.5, 155.6, 151.0, 124.6 (d, ³J_{CF}=10 Hz), 123.6, 114.3, 113.9 (d, ³J_{CF}=10 Hz), 113.5 (d, ²J_{CF}=25 Hz), 106.4 (d, ²J_{CF}=26 Hz), 103.8, 103.4, 102.0; HRMS (ESI): m/z calcd for C₁₅H₈FO₄ [M+H]⁺ 271.0401. found 271.0402.

Compound 8k (from table 1):

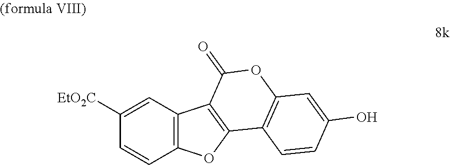 [SMILES: CCOC(=O)c1ccc2oc3c4ccc(O)cc4oc(=O)c3c2c1]

ethyl 2-(2,4-dimethoxyphenyl)-5-(trifluoromethyl)benzofuran-3-carboxylate (7k) (197 mg, 0.5 mmol) was treated with BBr₃ (2 mL, 2 mmol) according to general method C affording compound 8k (136 mg, 84%) as a white solid. ¹H NMR (500 MHz, DMSO-d₆, ppm) δ 10.88 (s, 1H), 8.28 (d, J=1.5 Hz, 1H), 7.98 (dd, J=8.7, 1.7 Hz, 1H), 7.85 (d, J=8.7 Hz, 1H), 7.82 (d, J=8.6 Hz, 1H), 6.90 (dd, J=8.6, 2.0 Hz, 1H), 6.85 (d, J=2.0 Hz, 1H), 4.32 (q, J=7.1 Hz, 2H), 1.35 (t, J=7.1 Hz, 3H); ¹³C NMR (125 MHz, DMSO-d₆, ppm) δ 165.5, 162.7, 162.1, 157.5, 157.2, 155.8, 127.7, 127.3, 123.81, 123.77, 121.6, 114.4, 112.6, 103.8, 103.5, 101.9, 61.5, 14.6; IR (KBr): 3292.0, 1734.7, 1708.7 cm-1; HRMS (ESI): m/z calcd for C₁₈H₁₃O₆ [M+H]⁺ 325.0707. found 325.0708.

Compound 8 (from table 1)l:

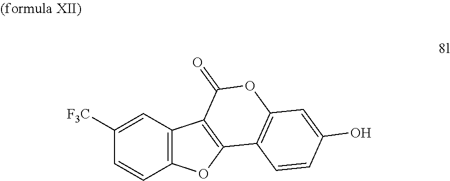 [SMILES: O=c1oc2cc(O)ccc2c2oc3ccc(C(F)(F)F)cc3c12]

BBr₃ (2 ml, 2 mmol) was added drop-wise into a stirred solution of ethyl 2-(2,4-dimethoxyphenyl)-5-(trifluoromethyl)benzofuran-3-carboxylate (7k) (197 mg, 0.5 mmol) in DCM under nitrogen atmosphere at 0° C. Reaction mixture was further stirred overnight at room temperature. Quenched with aq. NaHCO₃ (1 ml) and extracted with EtOAc (3×10 mL), dried over Na₂SO₄. The solvent was removed under reduced pressure. The residue was refluxed in toluene (5 ml) in the presence of Et₃N (0.5 eq) for 30 minutes. The solvent was removed under reduced pressure and the residue was purified by flash column chromatography on silica gel affording compound 8l (147 mg, 92%) as a white solid. ¹H NMR (400 MHz, DMSO-d₆, ppm) δ 7.78 (s, 1H), 7.77 (d, J=9.4 Hz, 1H), 7.61 (d, J=8.7 Hz, 1H), 7.61 (d, J=8.7 Hz, 1H), 7.78 (dd, J=8.7, 2.0 Hz, 1H), 6.69 (d, J=1.9 Hz, 1H); ¹³C NMR (100 MHz, DMSO-d₆, ppm) δ 162.9, 162.4, 157.3, 156.3, 155.9, 126.5 (q, ²J_{CF}=31 Hz), 124.6 (q, ¹J_{CF}=272 Hz), 124.1, 123.8, 123.6 (q, ³J_{CF}=4 Hz), 117.4 (q, ³J_{CF}=3 Hz), 114.5, 113.4, 103.6, 103.5, 101.7; HRMS (ESI): m/z calcd for C₁₆H₈F₃O₄ [M+H]⁺ 321.0369. found 321.0368.

Compound 8m (from table 1):

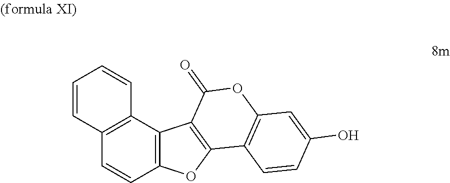 [SMILES: O=c1oc2cc(O)ccc2c2oc3ccc4ccccc4c3c12]

ethyl 2-(2,4-dimethoxyphenyl)naphtho[2,1-b]furan-1-carboxylate (7l) (181 mg, 0.5 mmol) was treated with BBr₃ (2 mL, 2 mmol) according to general method C affording compound 8m (101 mg, 67%) as a white solid. ¹H NMR (400 MHz, DMSO-d₆, ppm) δ 10.80 (br s, 1H), 9.51 (d, J=8.2 Hz, 1H), 8.07 (d, J=7.9 Hz, 1H), 8.02 (d, J=9.1 Hz, 1H), 7.96 (d, J=9.0 Hz, 1H), 7.93 (d, J=8.5 Hz, 1H), 7.69 (ddd, J=7.6, 6.9, 1.0 Hz, 1H), 7.58 (ddd, J=7.5, 6.8, 1.0 Hz, 1H), 6.96 (dd, J=8.5, 2.2 Hz, 1H), 6.93 (d, J=2.1 Hz, 1H), 4.32 (q, J=7.1 Hz, 2H), 1.35 (t, J=7.1 Hz, 3H); ¹³C NMR (100 MHz, DMSO-d₆, ppm) δ 162.1, 160.6, 158.5, 155.3, 152.8, 131.5, 129.2, 128.4, 127.4, 127.3, 126.7, 126.1, 123.5, 118.9, 114.4, 112.5, 104.29, 104.25, 103.1; HRMS (ESI): m/z calcd for C₁₉H₁₀O₄ [M+H]⁺ 303.0651. found 303.0648.

### Example 5

### Synthesis of Ethyl 2-(2-Methoxyphenyl)Benzofuran-3-Carboxylate (7B)

Ethyl 2-(2-methoxyphenyl)benzofuran-3-carboxylate (7b (from table 1)): ethyl 3-(2-methoxyphenyl)-3-oxopropanoate (222 mg, 1 mmol) and phenol (103 mg, 1.1 mmol) were coupled according to general procedure. The reaction mixture was heated to 70° C. for 8 h. The crude residue was purified (ethyl acetate-hexanes, 1:9) affording compound 7b (216 mg, 73%) as a white solid. ¹H NMR (400 MHz, CDCl₃, ppm) δ8.05-8.13 (m, 1H), 7.52-7.62 (m, 2H), 7.48 (ddd, J=7.1, 6.8, 1.7 Hz, 1H), 7.33-7.40 (m, 2H), 7.39 (ddd, J=7.4, 7.4, 0.8 Hz, 1H), 7.31 (d, J=8.3 Hz, 1H), 4.3 (q, J=7.4 Hz, 2H), 3.86 (s, 3H), 3.82 (s, 3H), 1.28 (t, J=7.2 Hz, 3H); ¹³C NMR (100 MHz, CDCl₃, ppm) δ 163.8, 158.1, 157.6, 154.1, 131.5, 131.3, 126.6, 124.8, 123.7, 121.9, 120.1, 119.4, 111.1, 111.0, 60.2, 55.5, 14.1; HRMS (ESI): m/z calcd for C₁₈H₁₇O₄ [M+H]⁺ 297.1121. found 297.1121.

### Example 6

### Synthesis of Ethyl 2-(2,4-Dimethoxyphenyl)-5,6-Dimethoxybenzofuran-3-Carboxylate (7C)

Ethyl 2-(2,4-dimethoxyphenyl)-5,6-dimethoxybenzofuran-3-carboxylate (7c (from table 1)): Ethyl 3-(2,4-dimethoxyphenyl)-3-oxopropanoate (252 mg, 1 mmol) and 3,4-dimethoxyphenol (170 mg, 1.1 mmol) were coupled according to general procedure. The reaction mixture was heated to 70° C. for 8 h. The crude residue was purified (ethyl acetate-hexanes, 2:8) affording compound 7c (205 mg, 53%) as a brown solid. ¹H NMR (400 MHz, CDCl₃, ppm) δ 7.49 (s, 1H), 7.45 (d, J=8.5 Hz, 1H), 7.05 (s, 1H), 6.58 (dd, J=8.3, 2.2 Hz, 1H), 6.53 (d, J=2.2 Hz, 1H), 4.27 (q, J=7.1 Hz, 2H), 3.97 (s, 3H), 3.92 (s, 3H), 3.86 (s, 3H), 3.78 (s, 3H), 1.26 (t, J=7.1 Hz, 3H); ¹³C NMR (100 MHz, CDCl₃, ppm) δ 164.2, 162.4, 158.9, 157.2, 148.7, 148.1, 147.1, 132.2, 118.9, 112.5, 110.5, 104.3, 102.9, 98.6, 95.0, 60.1, 56.3, 56.2, 55.6, 55.4, 14.2; HRMS (ESI): m/z calcd for C₂₁H₂₃O₇ [M+H]⁺ 387.1438. found 387.1422.

### Example 7

### Synthesis of Ethyl 6-Methoxy-2-(2-Methoxyphenyl)Benzofuran-3-Carboxylate (7D (from Table 1))

Ethyl 6-methoxy-2-(2-methoxyphenyl)benzofuran-3-carboxylate (7d (from table 1)): ethyl 3-(2-methoxyphenyl)-3-oxopropanoate (222 mg, 1 mmol) and 3-methoxy phenol (136 mg, 1.1 mmol) were coupled according to general procedure. The reaction mixture was heated to 70° C. for 12 h. The crude residue was purified (ethyl acetate-hexanes, 2:8) affording compound 7d (180 mg, 55%) as yellow oil. ¹H NMR (400 MHz, CDCl₃, ppm) δ 7.91 (d, J=8.6 Hz, 1H), 7.54 (dd, J=7.6 and 1.7 Hz, 1H), 7.45 (ddd, J=8.6, 7.3 and 1.8 Hz, 1H), 7.03-7.08 (m, 2H), 6.96-7.01 (m, 2H), 4.28 (q, J=7.1 Hz, 2H), 3.86 (s, 3H), 3.81 (s, 3H), 1.26 (t, J=7.1 Hz, 3H); ¹³C NMR (100 MHz, CDCl₃, ppm) δ 163.9, 158.3, 157.5, 157.2, 155.1, 131.3 (3 carbons), 122.2, 120.1, 119.9, 119.5, 112.7, 111.0, 95.6, 60.2, 55.7, 55.6, 14.1; HRMS (ESI): m/z calcd for C₁₉H₁₈O₅ [M+H]⁺ 327.1236. found 327.1223.

### Example 8

### Synthesis of Ethyl 2-(2,4-Dimethoxyphenyl)Benzofuran-3-Carboxylate (7E (from Table 1))

Ethyl 2-(2,4-dimethoxyphenyl)benzofuran-3-carboxylate (7e (from table 1)): Ethyl 3-(2,4-dimethoxyphenyl)-3-oxopropanoate (252 mg, 1 mmol) and phenol (103 mg, 1.1 mmol) were coupled according to general procedure. The reaction mixture was heated to 70° C. for 12 h. The crude residue was purified (ethyl acetate-hexanes, 2:8) affording compound 7e (251 mg, 77%) as yellow oil. ¹H NMR (400 MHz, CDCl₃, ppm) [symbol] 8.00-8.05 (m, 1H), 7.48-7.54 (m, 2H), 7.30-7.35 (m, 2H), 6.61 (dd, J=8.5 and 2.3 Hz, 1H), 6.56 (d, J=2.3 Hz, 1H), 4.32 (q, J=7.3 Hz, 2H), 3.87 (s, 3H), 3.79 (s, 3H), 1.30 (t, J=7.3 Hz, 3H); ¹³C NMR (100 MHz, CDCl₃, ppm) δ 164.0, 162.6, 159.0, 158.5, 154.0, 132.3, 126.8, 124.6, 123.6, 121.9, 112.1, 111.1, 110.5, 104.4, 98.6, 60.1, 55.6, 55.4, 14.2; HRMS (ESI): m/z calcd for C₁₉H₁₈O₅ [M+H]⁺ 327.1236. found 327.1229.

### Example 9

### Synthesis of Ethyl 2-(2,4-Dimethoxyphenyl)-5-Methoxybenzofuran-3-Carboxylate (7F (from Table 1))

Ethyl 2-(2,4-dimethoxyphenyl)-5-methoxybenzofuran-3-carboxylate (7f (from table 1)): Ethyl 3-(2,4-dimethoxyphenyl)-3-oxopropanoate (252 mg, 1 mmol) and 4-methoxy phenol (136 mg, 1.1 mmol) were coupled according to general procedure. The reaction mixture was heated to 70° C. for 8 h. The crude residue was purified (ethyl acetate-hexanes, 2:8) affording compound 7f (206 mg, 58%) as a brown solid. ¹H NMR (400 MHz, CDCl₃, ppm) δ 7.52 (d, J=2.6 Hz, 1H), 7.48 (d, J=8.4 Hz, 1H), 7.39 (d, J=8.8 Hz, 1H), 6.92 (dd, J=8.7, 2.6 Hz, 1H), 6.59 (dd, J=8.5, 2.3 Hz, 1H), 6.54 (d, J=2.3 Hz, 1H), 4.29 (q, J=7.1 Hz, 2H), 3.89 (s, 3H), 3.87 (s, 3H), 3.78 (s, 3H), 1.27 (t, J=7.7 Hz, 3H); ¹³C NMR (100 MHz, CDCl₃, ppm) δ 164.1, 162.6, 159.1, 158.9, 156.6, 149.0, 132.2, 127.5, 113.5, 112.3, 111.6, 110.5, 104.3, 104.0, 98.5, 60.1, 55.9, 55.5, 55.4, 14.2; HRMS (ESI): m/z calcd for C₂₀H₂₁O₆ [M+H]⁺ 357.1343. found 357.1320.

### Example 10

### Synthesis of Ethyl 6-Acetamido-2-(2,4-Dimethoxyphenyl)Benzofuran-3-Carboxylate (7G (from Table 1))

Ethyl 6-acetamido-2-(2,4-dimethoxyphenyl)benzofuran-3-carboxylate (7g (from table 1)): Ethyl 3-(2,4-dimethoxyphenyl)-3-oxopropanoate (252 mg, 1 mmol) and N-(3-hydroxyphenyl)acetamide (166 mg, 1.1 mmol) were coupled according to general procedure. The reaction mixture was heated to 70° C. for 16 h. The crude residue was purified (ethyl acetate-hexanes, 6:4) affording compound 7g (241 mg, 63%) as a white solid. ¹H NMR (400 MHz, CDCl₃, ppm) δ 8.1 (br s, 1H), 8.06 (d, J=1.1 Hz, 1H), 7.85 (d, J=8.2 Hz, 1H), 7.46 (d, J=8.7 Hz, 1H), 7.17 (dd, J=8.5, 1.7 Hz, 1H), 6.56 (dd, J=8.5, 2.1 Hz, 1H), 6.50 (d, J=2.1 Hz, 1H), 4.29 (q, J=7.1 Hz, 2H), 3.83 (s, 3H), 3.76 (s, 3H), 2.14 (s, 3H), 1.28 (t, J=7.1 Hz, 3H); ¹³C NMR (100 MHz, CDCl₃, ppm) δ 168.7, 164.1, 162.6, 158.8, 158.6, 154.1, 135.3, 132.2, 123.0, 121.5, 116.2, 111.9, 110.2, 104.4, 103.2, 98.5, 60.2, 55.5, 55.4, 24.4, 14.2; IR (KBr): 3340.2, 1697.1, 1612.2, 1596.8 cm-1; HRMS (ESI): m/z calcd for C₂₁H₂₂NO₆ [M+H]⁺ 384.1454. found 387.1435.

### Example 11

### Synthesis of Ethyl 5-Acetamido-2-(2,4-Dimethoxyphenyl)Benzofuran-3-Carboxylate (7H (from Table 1))

Ethyl 5-acetamido-2-(2,4-dimethoxyphenyl)benzofuran-3-carboxylate 7h (from table 1): Ethyl 3-(2,4-dimethoxyphenyl)-3-oxopropanoate (252 mg, 1 mmol) and N-(4-hydroxyphenyl)acetamide (166 mg, 1.1 mmol) were coupled according to general procedure. The reaction mixture was heated to 70° C. for 16 h. The crude residue was purified (ethyl acetate-hexanes, 6:4) affording compound 7h (260 mg, 68%) as a brown solid. ¹H NMR (500 MHz, CDCl₃, ppm) δ 8.05 (s, 1H), 7.94 (br s, 1H), 7.51 (d, J=8.7 Hz, 1H), 7.46 (d, J=8.4 Hz, 1H), 7.39 (d, J=8.7 Hz, 1H), 6.57 (dd, J=8.0, 2.0 Hz, 1H), 6.52 (d, J=1.3 Hz, 1H), 4.27 (q, J=7.1 Hz, 2H), 3.84 (s, 3H), 3.76 (s, 3H), 2.18 (s, 3H), 1.28 (t, J=7.1 Hz, 3H); ¹³C NMR (100 MHz, CDCl₃, ppm) δ 168.9, 163.9, 162.7, 159.3, 158.9, 151.0, 133.9, 132.2, 127.1, 118.3, 113.6, 111.9, 111.1, 110.4, 104.5, 98.6, 60.3, 55.5, 55.4, 24.2, 14.2; HRMS (ESI): m/z calcd for C₂₁H₂₂NO₆ [M+H]⁺ 384.1454. found 387.1440.

### Example 12

### Synthesis of Ethyl 5-Bromo-2-(2,4-Dimethoxyphenyl)Benzofuran-3-Carboxylate (71 (from Table 1))

Ethyl 5-bromo-2-(2,4-dimethoxyphenyl)benzofuran-3-carboxylate (7i (from table 1)): Ethyl 3-(2,4-dimethoxyphenyl)-3-oxopropanoate (252 mg, 1 mmol) and 4-bromo phenol (189 mg, 1.1 mmol) were coupled according to general procedure. The reaction mixture was heated to 70° C. for 24 h. The crude residue was purified (ethyl acetate-hexanes, 4:6) affording compound 7i (262 mg, 65%) as off white solid. ¹H NMR (400 MHz, CDCl₃, ppm) [symbol] 8.15 (s, 1H), 7.48 (d, J=8.5 Hz, 1H), 7.33-7.45 (m, 2H), 6.59 (d, J=8.6 Hz, 1H), 6.54 (s, 1H), 4.31 (q, J=7.4 Hz, 2H), 3.87 (s, 3H), 3.79 (s, 3H), 1.30 (t, J=7.1 Hz, 3H); ¹³C NMR (100 MHz, CDCl₃, ppm) δ163.5, 162.9, 159.6, 159.0, 152.7, 132.2, 128.7, 127.5, 124.6, 116.9, 112.5, 111.5, 109.9, 104.5, 98.6, 60.4, 55.5, 55.4, 14.2; HRMS (ESI): m/z calcd for C₁₉H₁₈BrO₅[M+H]⁺ 405.0332. found 405.0333.

### Example 13

### Synthesis of Ethyl 2-(2,4-Dimethoxyphenyl)-5-Fluorobenzofuran-3-Carboxylate (7J (from Table 1))

Ethyl 2-(2,4-dimethoxyphenyl)-5-fluorobenzofuran-3-carboxylate (7j (from table 1)): Ethyl 3-(2,4-dimethoxyphenyl)-3-oxopropanoate (252 mg, 1 mmol) and 4-fluoro phenol (123 mg, 1.1 mmol) were coupled according to general procedure. The reaction mixture was heated to 70° C. for 16 h. The crude residue was purified (ethyl acetate-hexanes, 4:6) affording compound 7j (251 mg, 73%) as a white solid. ¹H NMR (400 MHz, CDCl₃, ppm) δ 7.68 (dd, J=9.1, 2.6 Hz, 1H), 7.48 (d, J=8.5 Hz, 1H), 7.43 (dd, J=8.9, 4.1 Hz, 1H), 7.04 (ddd, J=9.1, 8.9, 2.6 Hz, 1H), 6.60 (dd, J=8.5, 2.3 Hz, 1H), 6.55 (d, J=2.3 Hz, 1H), 4.3 (q, J=7.1 Hz, 2H), 3.87 (s, 3H), 3.79 (s, 3H), 1.30 (t, J=7.2 Hz, 3H); ¹³C NMR (100 MHz, CDCl₃, ppm) δ 163.6, 162.8, 160.2, 159.8 (d, ¹J_{CF}=239 Hz), 159.0, 150.2, 132.2, 127.9 (d, ³J_{CF}=11.1 Hz), 112.3 (d, ²J_{CF}=26.3 Hz), 111.8 (d, ³J_{CF}=9.4 Hz), 111.8, 110.7 (d, ⁴J_{CF}=4.2 Hz), 107.7 (d, ²J_{CF}=26.2 Hz), 104.5, 98.6, 60.3, 55.6, 55.5, 14.2; HRMS (ESI): m/z calcd for C₁₉H₁₈FO₅ [M+H]⁺ 345.1132. found 345.1127.

### Example 14

### Synthesis of Ethyl 2-(2,4-Dimethoxyphenyl)-5-(Trifluoromethyl)Benzofuran-3-Carboxylate (7K (from Table 1))

Ethyl 2-(2,4-dimethoxyphenyl)-5-(trifluoromethyl)benzofuran-3-carboxylate 7k (from table 1): Ethyl 3-(2,4-dimethoxyphenyl)-3-oxopropanoate (252 mg, 1 mmol) and 4-(trifluoromethyl)phenol (178 mg, 1.1 mmol) were coupled according to general procedure. The reaction mixture was heated to 70° C. for 24 h. The crude residue was purified (ethyl acetate-hexanes, 4:6) affording compound 7k (200 mg, 51%) as a white solid. ¹H NMR (400 MHz, CDCl₃, ppm) δ 8.32 (br s, 1H), 7.58 (br s, 1H), 7.58 (br s, 1H), 7.51 (d, J=8.5 Hz, 1H), 6.61 (dd, J=8.5, 2.3 Hz, 1H), 6.55 (d, J=2.3 Hz, 1H), 4.33 (q, J=7.2 Hz, 2H), 3.88 (s, 3H), 3.80 (s, 3H), 1.31 (t, J=7.1 Hz, 3H); ¹³C NMR (100 MHz, CDCl₃, ppm) δ 163.4, 163.0, 160.1, 159.1, 155.2, 132.3, 127.1, 126.3 (q, ²J_{CF}=31.6 Hz), 124.6 (q, ¹J_{CF}=272.1 Hz), 121.7 (d, ³J_{CF}=3.3 Hz), 119.7 (d, ³J_{CF}=3.1 Hz), 111.5, 111.3, 110.5, 104.5, 98.6, 60.5, 55.54, 55.47, 14.2; HRMS (ESI): m/z calcd for C₂₀H₁₈F₃O₅ [M+H]⁺ 395.1100. found 395.1098.

### Example 15

### Synthesis of Ethyl 2-(2,4-Dimethoxyphenyl)Naphtho[2,1-B]Furan-1-Carboxylate (7M (from Table 1))

Ethyl 2-(2,4-dimethoxyphenyl)naphtho[2,1-b]furan-1-carboxylate (7m (from table 1)): Ethyl 3-(2,4-dimethoxyphenyl)-3-oxopropanoate (252 mg, 1 mmol) and 2-naphthol (216 mg, 1.5 mmol) were coupled according to general procedure. The reaction mixture was heated to 70° C. for 8 h. The crude residue was purified (ethyl acetate-hexanes, 4:6) affording compound 7l (244 mg, 65%) as a white solid. ¹H NMR (400 MHz, CDCl₃, ppm) δ 8.90 (d, J=8.4 Hz, 1H), 7.94 (d, J=7.9 Hz, 1H), 7.76 (d, J=9.0 Hz, 1H), 7.66 (d, J=8.9 Hz, 1H), 7.64 (d, J=8.6 Hz, 1H), 7.60 (ddd, J=8.5, 6.8, 1.2 Hz 1H), 7.50 (ddd, J=8.6, 6.7, 1.0 Hz, 1H), 6.64 (dd, J=8.4, 2.3 Hz, 1H), 6.55 (d, J=2.2 Hz, 1H), 4.34 (q, J=7.2 Hz, 2H), 3.88 (s, 3H), 3.81 (s, 3H), 1.21 (t, J=7.2 Hz, 3H); ¹³C NMR (100 MHz, CDCl₃, ppm) δ 166.2, 162.2, 158.1, 154.6, 151.7, 131.1, 131.0, 128.7, 127.7, 126.3 (2 carbons), 124.9, 124.6, 120.9, 113.1, 112.9, 111.9, 104.6, 98.5, 60.8, 55.5, 55.4, 13.9; HRMS (ESI): m/z calcd for C₂₃H₂₁O₅ [M+H]⁺ 377.1383. found 377.1364.

What is claimed is:

1. A compound selected from any one of the formulae VII, IX, XIII, X, XI, XII, or a pharmaceutically acceptable salt thereof, wherein the compound represented by formula VII is: 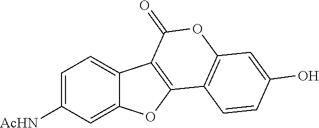 [SMILES: CC(=O)Nc1ccc2c(c1)oc1c3ccc(O)cc3oc(=O)c21]
the compound represented by formula IX is: 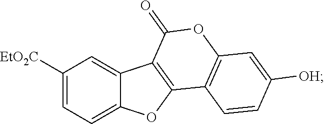 [SMILES: CCOC(=O)c1ccc2oc3c4ccc(O)cc4oc(=O)c3c2c1]
the compound represented by formula XIII is: 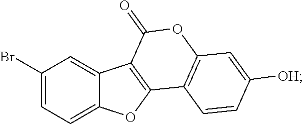 [SMILES: O=c1oc2cc(O)ccc2c2oc3ccc(Br)cc3c12]
the compound represented by formula X is: 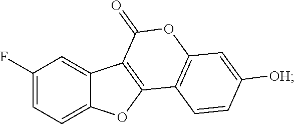 [SMILES: O=c1oc2cc(O)ccc2c2oc3ccc(F)cc3c12]
the compound represented by formula XI is: 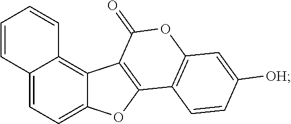 [SMILES: O=c1oc2cc(O)ccc2c2oc3ccc4ccccc4c3c12]
and
the compound represented by formula XII is:

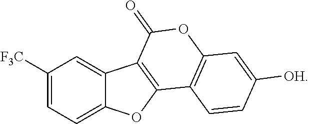 [SMILES: O=c1oc2cc(O)ccc2c2oc3ccc(C(F)(F)F)cc3c12]

2. (canceled)

3. (canceled)

4. (canceled)

5. (canceled)

6. (canceled)

7. (canceled)

8. A pharmaceutical composition comprising a compound according to claim 1 and a pharmaceutically acceptable excipient.

9. A method for inhibiting mitosis of an estrogen dependent cancer cell, comprising contacting said cell with a compound according to claim 1.

10. A method for treating a subject afflicted with any one of the following diseases or disorders: (i) an estrogen dependent cancer, (ii) enhanced bone turnover, or (iii) elevated cholesterol and triglycerides levels, the method comprising administering to said subject the pharmaceutical composition of claim 1.

11. The method of claim 10, wherein said estrogen dependent cancer is breast cancer.

12. (canceled)

13. The method of claim 10, wherein said enhanced bone turnover is postmenopausal osteoporosis.

14. (canceled)

15. A method for selectively modulating an estrogen receptor in a cell, comprising contacting said cell with a compound selected from the group consisting of: 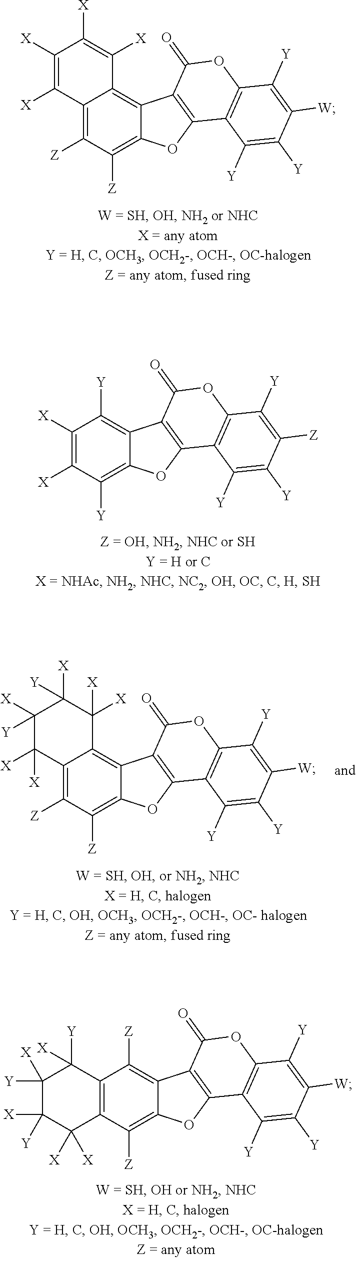
or a pharmaceutically acceptable salt thereof, thereby inhibiting mitosis of an estrogen dependent cancer cell.

16. (canceled)

17. (canceled)

18. (canceled)

19. The method of claim 15, wherein said selectively modulating an estrogen receptor in a cell is inhibiting mitosis of an estrogen dependent cancer cell.

20. The method of claim 15, wherein said selectively modulating is agonizing activity in a bone tissue.

21. The method of claim 15, wherein said selectively modulating is antagonising activity in a breast tissue.

22. The method of claim 19, wherein said estrogen dependent cancer is breast cancer cell.

23. A process for the preparation of a compound of formula I: 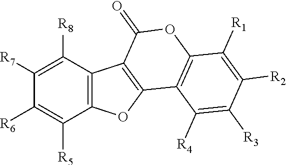
wherein:
R₁, R₃, R₄, R₅ and R₈ each independently represent H, C, or a halogen;
R₂ represents Oalkyl, OS(O)₂, OH, H, N or a halogen;
R₆ represents O, H, C, N or C(O)N, alkyl-NH, S(O)NH, AcNH; and
R₇ represents O, H, C, N, C(O)N, alkyl-NH, S(O)NH, AcNH, CO₂Et, CF₃ or a halogen;
said process comprising lactonization of a deprotected benzofuran of formula II: 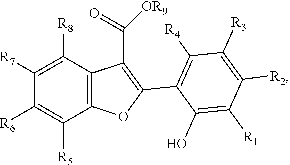
wherein:
R₁, R₃, R₄, R₅ and R₈ each independently represent H, C, or a halogen C;
R₂ represents Oalkyl, OS(O)₂, OH, H, N or a halogen;
R₆ represents O, H, C, N or C(O)N, alkyl-NH, S(O)₂NH, S(O)NH, or AcNH;
R₇ represents O, H, C, N, C(O)N, alkyl-NH, S(O)₂NH, S(O)NH, AcNH, CO₂Et, CF₃ or a halogen;
and R₉ represents H or C, CH₃, C₂H₅; thereby preparing a compound of formula I.

24. A process for the preparation of a compound of formula III: 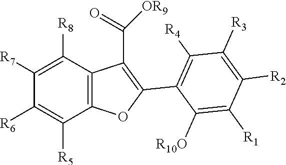
wherein:
R₁, R₃, R₄, R₅ and R₈ each independently represent H, C, or a halogen;
R₂ represents Oalkyl, OS(O)₂C, OH, H, N or a halogen;
R₆ represents O, H, C, N or C(O)N, alkylNH, S(O)₂NH, S(O)NH, AcNH; and
R₇ represents O, H, C, N, C(O)N, alkylNH, S(O)₂NH, S(O)NH, AcNH, CO₂Et, CF₃ or a halogen;
R₉ represents H or C, CH₃, C₂H₅; and
R10 represents C, S, Si;
said process comprising mixing ethyl 2-(2,4-dimethoxybenzoyl)acetate and 3-methoxyphenol in 1,2-dichloroethane in the presence of FeCl₃ under air atmosphere or oxygen atmosphere, thereby preparing a compound of formula III.

25. The process of claim 23, wherein said lactonization of a deprotected benzofuran is performed in a polar solvent or a non-polar solvent.

26. The process of claim 23, wherein said deprotected benzofuran is obtained by contacting a benzofuran of formula III: 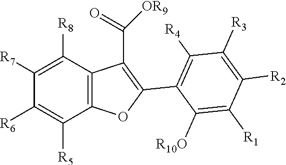
wherein:
R₁, R₃, R₄, R₅ and R₈ each independently represent H, C, or a halogen;
R₂ represents Oalkyl, OS(O)₂C, OH, H, N or a halogen;
R₆ represents O, H, C, N or C(O)N, alkylNH, S(O)₂NH, S(O)NH, AcNH; and
R₇ represents O, H, C, N, C(O)N, alkylNH, S(O)₂NH, S(O)NH, AcNH, CO₂Et, CF₃ or a halogen;
R₉ represents H or C, CH₃, C₂H₅;
R10 represents C, S, Si, with a deprotecting solution/agent.

27. The process of claim 26, wherein said benzofuran of formula III is obtained by iron catalyzed oxidative cross coupling reaction between a compound of formula IV: 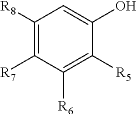
and a compound of formula V: 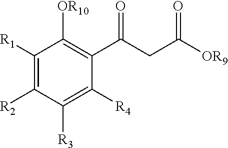
wherein:
R₁, R₃, R₄, R₅ and R₈ each independently represent H, C, or a halogen;
R₂ represents H, OMe, or a halogen;
R₆ represents OMe, H, C, N or AcNH;
R₇ represents OMe, H, C, N, AcNH, CO₂Et, CF₃ or a halogen;
R₉ represents C or H; and
R₁₀ represents H or C.

28. The process of claim 26, wherein said benzofuran of formula III is obtained by the process of claim 24.

29. A product comprising formula VI: 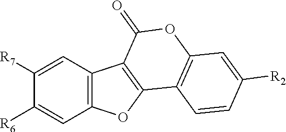
obtained by the process of claim 23, wherein:
R₂ represents OH;
R₆ represents H, or AcNH; and
R₇ represents H, AcNH, CO₂Et, F, or CF₃.

30. The product of claim 29, represented formula VII:

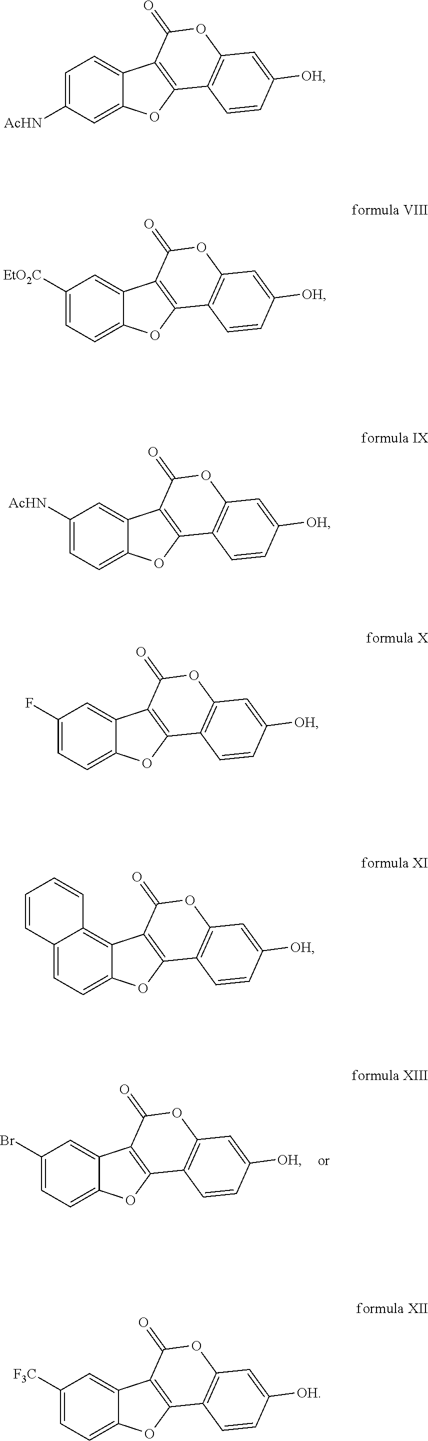

31. A pharmaceutical composition comprising a product according to claim 30 and a pharmaceutically acceptable excipient.

32. (canceled)
